(19) United States
(12) Patent Application Publication
(10) Pub. No.: US 2026/0287192 A1
(43) Pub. Date: Sep. 24, 2026

(54) ANTI-PATHOGENIC SYSTEM

(71) Applicant: AIR FORCE-FIELD SYSTEMS LLC, Chino Hills, CA (US)

(72) Inventor: Fredrick T Howard, Chino Hills, CA (US)

(21) Appl. No.: 19/688,962

(22) Filed: May 27, 2026

Related U.S. Application Data
(63) Continuation-in-part of application No. 19/007,392, filed on Dec. 31, 2024.
Application No. 19/007,392 is a continuation-in-part of application No. 18/111,498, filed on Feb. 17, 2023.
Application No. 18/111,498 is a continuation-in-part of application No. PCT/US21/54453, filed on Oct. 11, 2021.
(60) Provisional application No. 63/090,285, filed on Oct. 12, 2020.
(60) Provisional application No. 63/122,474, filed on Dec. 8, 2020.
(60) Provisional application No. 63/176,369, filed on May 6, 2021.
(60) Provisional application No. 63/194,890, filed on May 28, 2021.

(51) Int. Cl. F24F 9/00; A61G 7/05

(52) U.S. Cl. CPC F24F 9/00; A61G 7/05

(57) ABSTRACT

The disclosed system comprises a primary air curtain system and one or more optional apparel and/or accessory air curtain subsystem components. The primary system and subsystem(s) are configured to create a directionally controlled air curtain effect that repulses, repels, redirects, neutralizes, and/or disperses airborne infectious pathogens, carcinogens, chemicals, and other contaminants away from the body of a user or a surface susceptible to foreign contamination within a protected enclosure.

### CROSS-REFERENCE TO RELATED APPLICATIONS

The present application is a continuation-in-part of U.S. patent application Ser. No. 19/007,392, entitled “ANTI-PATHOGENIC SYSTEM,” filed Dec. 31, 2024, which is a continuation-in-part of U.S. patent application Ser. No. 18/111,498, entitled “ANTI-PATHOGENIC SYSTEM,” filed Feb. 17, 2023, which is a continuation-in-part of PCT Application No. PCT/US21/54453, entitled “ANTI-PATHOGENIC SYSTEM,” filed Oct. 11, 2021, which claims the benefit of U.S. Provisional Application No. 63/194,890, entitled “Anti-Contaminate Primary System Enclosure and Apparel Garment and Accessory Subsystem Extension,” filed May 28, 2021; U.S. Provisional Application No. 63/176,369, entitled “Anti-Contaminate Primary System Enclosure and Apparel Garment and Accessory Subsystem Extension,” filed May 6, 2021; U.S. Provisional Application No. 63/122,474, entitled “Anti-Contaminate Air Force-Field Enclosure And Body Worn Mobile Subsystem Facilitator,” filed Dec. 8, 2020; and U.S. Provisional Application No. 63/090,285, entitled “Anti-Contaminate Work Station Primary System and Utility Apparel Accessory Body Subsystem,” filed Oct. 12, 2020, the contents of each of which are hereby incorporated by reference herein in their entireties.

### TECHNICAL FIELD

The present disclosure relates to air curtain enclosures and air curtain apparel, garment, and accessory extensions.

### BACKGROUND

The Covid-19 pandemic has reinforced the continued need for anti-contaminant devices and maintainable sterile environments in homes, residential structures, buildings and work spaces. Anti-contaminant devices and systems are used to protect from various types of contaminants, such as smoke, allergens, pollen, dust, spores, toxic chemicals, undesirable odors, insects (e.g., gnats and mosquitoes), airborne pathogens (e.g., viruses that cause Covid-19, common colds, influenza, SARS, Ebola, tuberculosis, etc.), etc.

The first known record of an air curtain was in 1904. Although the terms may be used synonymously, in North America “air door” is more often referred to as an “air curtain.” Air curtains and air doors function differently from the present system.

Webster defines an air curtain as: an artificially created stream of air that is blown across a doorway or other opening to create a barrier (as for repelling insects or preventing heat transfer). The American Society of Heating, Refrigerating and Air-Conditioning Engineers (ASHRAE) defines air-curtains as follows: “In its simplest application an air curtain is a continuous broad stream of air circulated across a doorway of a conditioned space. It reduces penetration of insects and unconditioned air into a conditioned space by forcing an air stream across the entire entrance. The airstream moves with velocity and angle such that any air that tries to penetrate the curtain is entrained. Air curtain effectiveness in preventing infiltration through an entrance generally ranges from 60 to 80%.”

In 1888 Theophilus Van Kannel received a patent (U.S. Pat. No. 387,571) on the first air-door, also known as the revolving door. The purpose of Kannel's invention was to prevent cold air from the outside of a building from moving inside, and to also greatly reduce noise pollution. Kannel's system circulated air in a clockwise or counter-clockwise direction but was not designed for launching air mass away from a person's body or respiratory system.

Additionally, Kannel's system wasn't designed to protect individuals within a command control, utility, residential enclosure, or leisure area with wearable subsystem components of the parent system extending protection to individuals beyond the protective area.

Anti-contaminant and maintainable sterile environment dwellings and work spaces are the very foundation of human survival in everyday life and are as necessary as food and water to ensure survival from the elements. Some of these elements include: acidic rain, heat, cold, carcinogenic smoke, foul odors, allergens diseases, etc., as well as airborne pathogens that can also be transported by flying insects such as disease-carrying mosquitoes, flies, gnats, etc.

For example, even as far back as the pre-historic era, early men and women would shield themselves within various structures or formations from the elements by making their dwellings (including temporary residential structures and homes), work, and leisure areas, however crude, as environmentally breathable and habitable as possible. To escape the elements man would often construct their homes and work areas from fortifications such as caves, tents, cabins, tree houses, and eventually modern homes, expansive apartments and condominiums and large office buildings and skyscrapers.

And so, when ancient (and modern) men and women ventured from their obsolete dwellings and places of work, they found they were in needed of anti-contaminant extensions of protection from the semi-protective environments in which they had worked or lived, and which had afforded them centuries of protection. Some of these past and current contaminant-resistant extensions found in their homes, dwellings, or places of work have included sleep coverings, clothing apparel, and wearable accessories and garments to cover up their arms, hands, legs, and necks, as well as manual and electric lightweight material such as hand fans to disperse smoke, allergens, toxic odors, heat, swarming gnats, and mosquitos as well as body-wearable accessories to cover the eyes and mouth such as masks, scarves, eyeglasses, goggles, face shields, hats, boots, etc.

In addition to the aforementioned, men and women have gone to great lengths to ensure that their homes and work station environments are as disease free and environmentally safe as possible from over exposure to harmful airborne pathogens, chemicals, and carcinogens including natural and man-made substances such as: the Covid-19 virus (coronavirus), influenza viruses, cold viruses, aerosols, chemicals, pollen, smoke, toxic steam, disease-carrying insects, etc.

Other sterile work environments include scientific research facilities, as well as chemical plants and business transaction and workstations where employees are exposed to both long and short-term hazardous chemicals, airborne germs, and/or smoke.

These workstations may also take the form of traditional, hybrid, alternative fuel, AI enhanced or autonomous transportable and/or convertible tactical public safety mobile or ICS (Incident Command) headquarters vehicles such as crime scene investigation trailers, buses, patrol vehicles, SWAT vehicles, or other initial-response police and/or logistical law enforcement or support-related vehicles.

Additionally, large-scale mobile anti-contaminant traditional, hybrid, alternative fuel, AI-enhanced or autonomous ICS vehicles include forestry fighter trucks and rescue vehicles as well as urban fire truck and rescue ladder bucket vehicles, which may also be designated as initial-response short- or long-term command-and-control or incident command headquarter vehicles.

The air quality environments within these stationary and mobile command and control enclosures are often mandated by local, state, and/or federal laws and regulatory boards or commissions such as the EPA and OSHA as well as state and county departments of health and safety established to ensure environmentally safe compliance with mandates to prevent mass population infections and contamination outbreaks.

Traditional methods in the prior art for delivering a curtain of air across an opening of a doorway space are known in the art and are exemplified in Xiaofeng (Chinese Pat. CN2122530055U). Known air curtains primarily focus on protecting a doorway or barrier opening into a building or structure and disclose only slight innovations of the original purposes (i.e., protecting an entrance or exit).

For example, another variation of an anti-contaminant maintainable sterile environment relates to a smaller or miniature version of an air door system introduced in the art as an infant incubation system. This system utilizes an impeller contained within the incubator compartment of the enclosure which controls a precise temperature setting and humidity level decisive to the survivability of a premature or sick infant.

Like Kannel's system, a door or opening is also introduced which separates the sterile and protected environment within the incubator from the dangerous environment outside of the incubation enclosure.

When the door is breached, a curtain of air is introduced across the opening which prevents the escape of an optimum or prescribed temperature and humidity, while also reducing ingress of foreign contaminates or extremities into the enclosed compartment of the incubator.

One slight variation beyond the traditional air curtain is Xiaofeng (Chinese Pat. CN2122530055U). Xiaofeng teaches an air curtain machine that produces ozone, comprising a rack, fan, and a control device, wherein a cavity is formed within the rack. The rack is provided with an air inlet and an air outlet. An ozone generator is placed within the cavity for outputting ozone gas, thus disinfecting people as they pass through the air curtain.

Xiaofeng does not teach a wearable hardware system solution protective parent enclosure or subsystem extension. The method of deployment is unremarkable, in that Xiaofeng merely teaches the practicality of traditional air curtains but from the perspective of disinfection. Additionally, significant exposure to ozone generators has been shown to cause serious negative effects on a person's respiratory system and overall health without adequate filtration.

Xiaofeng also fails to teach a protective enclosure optionally expelling an air curtain from at least one to a plurality of directions away from a protective area or a person's body who is wearing at least one subcomponent of the parent systems around or attached to the person's body, nor does Xiaofeng cite using any wearable subcomponents as extensions beyond the protective area for the purpose of preventing cross contamination and activating a warning system upon reentering a protective area should a communication algorithm linking the systems be disrupted.

Additionally, because Xiaofeng is based on the traditional air curtain model, with the exception of employing a rack and cavity system for generating ozone, Xiaofeng also fails to disclose that its area of protection can also be an enclosure within a motor-driven or mobile command and control, work, utility, experimental, or leisure area. As such, the functionality of Xiaofeng is unrelated in scope to the purpose and intent of the proposed system.

Korean Pat. KR20010022820A relates to a system for face and respiratory protection. Unlike the present system, KR20010022820A proposes a system primarily designed for medical settings by applying a method of comfort and ease of use for the wearer while donning the system.

KR20010022820A proposes a face protection system wherein the face protector and the wearer's face substantially form a breathing area within the mask. A bag-shaped filter surrounds the outside of the blower and provides filtered air to the breathing area, and the exhaust filter is coupled with a face guard.

KR20010022820A cites the benefit of protecting the user from contamination while also protecting the environment (including non-wearers) from the user to include protecting patients. Additionally, KR20010022820A describes a face mask and shield system which form fits around the face and is anchored to the top portion of the head for increased stability without the necessity of traditional straps around the head.

While perhaps a solution in terms of comfort and to mutually safeguard anti-contaminant environments for individuals and professionals, (i.e., doctors and patients), KR20010022820A does not teach a wearable hardware system solution protective parent enclosure or subsystem extension to prevent, deter, or reduce the negative effects of contaminants, airborne carcinogens, pathogens, and allergens.

KR20010022820A also fails to teach a protective enclosure expelling an air curtain from at least one to a plurality of directions away from a protective area or a person's body who is wearing at least one subcomponent of the parent system around or attached to the person's body, nor does KR20010022820A cite using any wearable subcomponents as extensions beyond the protective area for preventing cross contamination upon reentering the protective area.

Unlike KR20010022820A, the present system is designed to protect a person in the course of performing his/her duties from contaminated environments such as battlefield operating rooms, forestry fire mobile and stationary command centers, or law enforcement ICM staging areas where gases or smoke may be deployed, etc. Neither KR20010022820A nor Xiaofeng protect the environment or person(s) outside of the protective enclosure.

The disclosed system offers several very different solutions from KR20010022820A and Xiaofeng, e.g., that it deploys a curtain of air directly away from a person while within an enclosure. It then extends the protective air curtain beyond the enclosure by equipping an individual with apparel and apparel attachments, garments and garment attachments, and accessories and accessory attachments that communicate with the parent system to alert or warn the wearer to prevent or reduce occurrences and epidemics of cross and mass contamination. Neither KR20010022820A nor Xiaofeng disclose such a system.

Russell (U.S. Pat. No. 7,077,137B2) introduces as a system for filtering, diffusing, and conditioning inhaled air. The device is attached to an eyewear system for convenience of use and includes air permeable membranes surrounding the nose for normal breathing and is easily removed and suitable for daily use. Russell describes a method of convenience relating to delivering filtered air into an individual's respiratory system via an eyewear system attachment. However, the proposed system, more specifically the eyewear subsystem, delivers a force field curtain of air mass forward or away from the body, which is not disclosed in Russell.

Decontamination chambers are also known in the art and are generally predicated upon the sterilization of articles such as medical supplies and personnel working in high contaminant environments subject to exposure. These areas may include places such as chemical factories as well as biological and nuclear testing and experimentation facilities. These chambers work along similar design principles, primarily involving the following steps:
A. The person removes a piece of equipment or clothing from a sterile environment.
B. The person or equipment is donned or prepared outside of the sterile environment but usually in a safe environment.
C. The person or equipment is introduced into a non-sterile or contaminated environment to complete a task or serve a purpose.
D. The person or equipment is removed from the non-sterile or contaminated environment and the environment is evacuated of the contaminants to the greatest extent possible.
E. If the person or equipment is removed from the environment the person or equipment is introduced into a chamber or wash setting and the chamber is secured.
F. A decontamination agent is introduced into the setting and the person or equipment is decontaminated or sterilized by various means practiced in the art.
G. The person or equipment is returned to a sterile environment to the extent possible or to an environment free of the harmful agents.

Wiget (WO 2014165428A) introduces a transportable vacuum assisted decontamination unit comprising a housing which contains a decontamination chamber and a decontamination processing section, and a master pallet on which the housing is positioned. Wiget's system also has at least one gas inlet and at least one gas outlet for importing and evacuating gaseous decontaminant agents.

However, none of what Wiget proposes indicates a primary system whereby the system exports mass matter away from an occupied enclosure nor does Wiget mention any integration with any subsystem components of the parent system in communication with a least one of the subsystem components to introduce a decontamination alert system.

Johnson (U.S. Pat. No. 10,577,744B2) teaches a fabric with a contaminant resistant nanoparticle coating that is applied during the fabrication process. Unlike the present invention, Johnson does not teach an air curtain system or device that utilizes exported air or other mass matter to repel airborne contaminates away from a wearer.

Additionally, Johnson fails to teach a system wherein clothing, apparel, garments, and/or accessories are integrated with a primary system or device and are in communication with the primary system to alert of a decontamination event.

Shankman (U.S. Pat. No. 4,858,256A) teaches a chemical equipment decontamination truck for man-carried equipment washing down personnel at a major chemical disaster. The vehicle contains equipment for decontaminating the equipment used by emergency personnel so that the man-carried equipment can be brought back into service quickly. Additionally, Shankman proposes that contaminated liquids are retained in the truck for disposition or are decontaminated before being returned to the environment.

Shankman also discloses a transportation means, specifically a truck, that is compartmentalized for various processes of decontamination. However, none of Shankman's aforementioned processes would suggest to one of ordinary skill in the art a vehicle as a protective enclosure expelling an air curtain from at least one to a plurality of directions away from the truck or a protective area or a person's body who is wearing at least one subcomponent of the parent systems around or attached to the person's body, nor does Shankman cite using any wearable subcomponents as extensions beyond the protective area for the purpose of preventing cross contamination upon reentering the truck vehicle.

Shankman's invention also fails to propose a mobile command-and-control system or method to import or export any mass or volume substance away from its enclosure or perimeter to protect personnel who are contained inside.

Therefore, Shankman's disclosure is fundamentally different from the present system, because Shankman's objective is to contain and decontaminate personnel and items within its enclosure while a purpose of the disclosed system is to expel airborne decontaminants away from an enclosure or person.

There is currently a need in the art for a more flexible and protective anti-contaminant system, that includes an anti-contaminant protective enclosure and one or more subsystem extensions to prevent, deter, or reduce contaminants, e.g., carcinogens, pathogen, and/or allergens.

The prior art does not teach a supplemental family of personnel protective equipment (PPE) extensions on a person, animal, or other living organism (e.g., a plant) beyond a contained command and control, work, utility, experimental, medical, or leisure area via the application of body-worn apparel, garments, harnesses or portably transportable subsystem accessory components. Additionally, the prior art does not disclose the aforementioned PPE extensions in communication with a primary system enclosure and/or at least one optionally programmable, secondary primary system enclosure (optionally functioning as a subsystem component) to signal a decontamination event upon egress or ingress beyond, within, or proximate to the protective enclosure.

The prior art does not teach a stationary or mobile medical bed, infant incubation bed, infant bassinet bed, patient treatment bed, bariatric bed, or medical procedure platform which may operate independently of a primary system.

The prior art does not teach the aforementioned primary system being programmable to facilitate integration with a supplemental family of wearable anti-pathogenic, personal protective equipment receptive to electronic, digital, programmed or preprogrammed instructions from at least one integrated anti-pathogenic bed system.

The prior art also does not introduce an anti-pathogenic bed or bed enclosure capable of delivering an ionization current or other contaminant neutralizer to protect a contained person, animal, or other living organisms from airborne contaminants.

The prior art does not introduce an anti-pathogenic bed system integrated with at least one anti-pathogenic subsystem device which exports mass away from or across a person, animal, or living organism within a residential, medical, scientific, or research facility bed.

The prior art also does not introduce an anti-pathogenic traditional, hybrid, alternative fuel, AI enhanced, or autonomous vehicle in communication with at least one wearable anti-pathogenic subsystem or mobile device (e.g., cell phone, tablet, laptop, etc.) platform for the purpose of cross system notification upon detecting that a protected person or location within a house, building, medical complex, government installation, etc., has been exposed to pathogens or other harmful contaminants.

In addition to the aforementioned, the prior art does not disclose an anti-pathogenic residential or commercial building enclosure or structure having a pathogen receiver or identifier disposed on the structure, preferentially adjacent to a door (similar to a doorbell notification), and whereby entry into the building structure requires a person to cough, blow, or otherwise expel respiratory air onto at least one pathogen receiver, and whereby the anti-pathogenic system detects pathogens in the air sample and notifies at least one subsystem component of the results so that the person can be granted or denied entry into the structure.

In addition, the prior art does not disclose an anti-pathogenic audio-visual, building structure access monitoring system integrated with a pathogen receiver or identifier (a pathogen type doorbell) to prevent or deter infected people from entering a residential home or other building structure.

The prior art also does not disclose any of the aforementioned processes and systems further including protective extensions (subsystems), predicated upon an individual donning or attaching a wearable or portably transportable air curtain system or device component that pushes exported mass matter, away from or across at least one direction of a person's body.

In addition to the aforementioned, the prior art fails to teach an anti-contaminant system whereby each distal component of the system can detect airborne contaminates by employing bio sensors in conjunction with AI and machine learning.

Additionally, the prior art does not disclose a wearable or transportable protective anti-pathogenic extension integrated with a motion detection or other machine learning means such as: an accelerometer, FPGA and/or ASIC assisted learning implementation chip, computer program, AI implemented program or other communication means known in the art/that can sense, detect, convert, mimic, read, translate, predict motion, recognize sound, speech, sign language, body repetitions, facial recognitions, temperatures or bioactivity, proximate to the subsystem (or primary system) for the purpose of relaying the translation and/or conversion back to at least one additional or optionally integrated parent system and/or at least one additional or optionally integrated anti-pathogenic primary system or subsystem component device.

### SUMMARY

In accordance with the foregoing objectives and others, exemplary methods and systems are disclosed herein to provide an anti-contaminant system. The disclosed methods and systems provide an anti-contaminant and anti-pathogenic air-force-field curtain barrier designed for work utility protection, fire and EMS protection, fire response and rescue (e.g., structure, forestry, etc.) protection, law enforcement and public safety protection, infant protection, as well as for anti-contaminant medical and respiratory pediatric and geriatric preventive care treatment. The system may also assist in preventing airborne contaminants and pathogenic carrying insects from contacting immune deficient patients on respirators or other life support systems in medical environments.

In general, the disclosed system comprises a primary air curtain system that protects a protected area, and one or more apparel and/or accessory air curtain subsystem components that are worn by people who are inside or in proximity to the protected area. The primary system and subsystem(s) are configured to create one or more directionally controlled air curtain effects that repulse, repel, redirect, neutralize, and/or disperse airborne infectious pathogens, carcinogens, chemicals, and other contaminants away from the body of subsystem users or a surface susceptible to contamination within a protected enclosure. The propulsion mechanism for the disclosed system may employ standard fan systems, bladeless fan systems, brushless fan systems or impeller centrifugal fan technology systems.

The primary system may comprise one or more devices that expel mass, such as air or water, from within the protected area. The protected area may be a work, command, or leisure enclosure; a specialized sterile environment (such as a manufacturing facility); a classroom; a mobile command platform (e.g., firetruck); or any other area from which there is a need to remove contaminants.

The primary system and the subsystem(s) may act together or independently of each other, and may be linked by communication methods known in the prior art. In an embodiment, the communication (or lack thereof) between the primary system and the subsystem(s) may signal the need for a decontamination event.

One embodiment is directed to an anti-pathogenic docking containment system comprising: a structural frame defining a containment region positionable relative to an occupant support surface; and at least one airflow generation subsystem operatively associated with the structural frame and configured to emit airflow outward along at least one boundary of the containment region to form a protective airflow boundary around at least a portion of the occupant support surface; wherein the docking containment system is operable independently of the occupant support surface; wherein the structural frame is configurable for integration into, mounting to, or positioning relative to a wall structure, architectural structure, room structure, building structure, mobile platform, or vehicle structure; and wherein the airflow generation subsystem is configured to emit laminar airflow, turbulent airflow, directional airflow, directional airflow of adjustable trajectory, or combinations thereof.

Another embodiment is directed to an anti-pathogenic modular containment apparatus comprising: a structural module configured to be integrated with, attached to, or detachable from a bed or occupant support surface; and an airflow generation subsystem associated with the structural module and configured to emit outward airflow to resist encroachment of airborne contaminants; wherein the structural module is reconfigurable between a bed-associated configuration and a remote-mounted configuration while maintaining airflow containment capability; and wherein the structural module is configured for articulated, pivotable, translatable, sliding, rotational, angular repositioning, or combinations thereof relative to the occupant support surface.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an embodiment of a system according to an aspect of the present invention.

FIG. 2 illustrates an example primary system within a medical research facility.

FIG. 3 illustrates an example primary system device.

FIG. 4 illustrates an example subsystem device.

FIG. 5 illustrates a cutaway of an example subsystem device.

FIG. 6 illustrates an example of eyewear with attached subsystem devices.

FIG. 7 illustrates an example of use of eyewear with attached subsystem devices.

FIG. 8 illustrates an example of an armguard with integrated subsystem devices.

FIG. 9 illustrates an example of use of an armguard with integrated subsystem devices.

FIG. 10 illustrates an example of an infant carrier with integrated primary system devices.

FIG. 11 illustrates an example of an infant carrier with integrated primary system devices.

FIG. 12 illustrates a second example of an infant carrier with integrated primary system devices.

FIG. 13 illustrates a second example of an infant carrier with integrated primary system devices.

FIG. 14 illustrates an example of a firetruck with integrated primary system devices.

FIG. 15 illustrates an example of a firetruck with integrated primary system devices.

FIG. 16 illustrates an example of a fire ladder with integrated primary system devices.

FIG. 17 illustrates an example of a primary system anti-pathogenic residential home.

FIG. 18 illustrates an example of an anti-pathogenic bed.

FIG. 19 illustrates another example of an anti-pathogenic bed.

FIG. 20 illustrates an example of an anti-pathogenic filament intake receiver pad.

FIG. 21 illustrates a man who has requested entry into an anti-pathogenic protected house.

FIG. 22 illustrates a man blowing onto a receiver pad.

FIG. 23 illustrates a man entering a home after being allowed entry.

FIG. 24 illustrates a bio-sensor that detects pathogens and other contaminants in a vehicle.

FIG. 25 illustrates an example digital dashboard display of a vehicle.

FIG. 26 illustrates an audio-video monitoring system.

FIGS. 27, 27A, 28, 28A, and 29 illustrate embodiments of an anti-pathogenic glove.

FIGS. 30, 31, 32, 33, 34, 35, 36 illustrate use cases of an anti-pathogenic glove.

FIG. 37 illustrates an anti-pathogenic receiver pad.

FIGS. 38 and 39 illustrate another embodiment of an anti-pathogenic glove.

FIGS. 40 and 41 illustrate additional use cases of an anti-pathogenic glove.

FIGS. 42, 43, and 44 illustrate additional embodiments of anti-pathogenic gloves.

FIG. 45 illustrates internal components of an embodiment of an anti-pathogenic glove.

FIG. 46 illustrates an embodiment of a dockable containment structure.

FIG. 47 illustrates an embodiment of a dockable containment structure.

### DETAILED DESCRIPTION

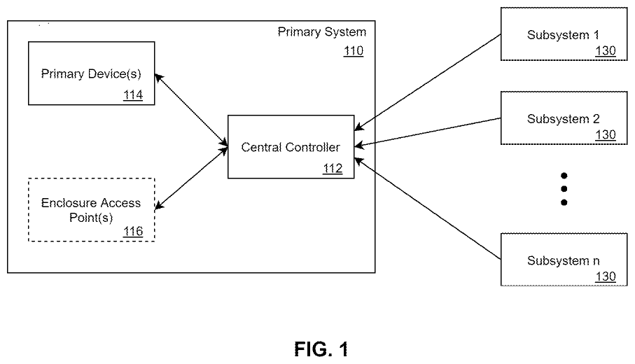

Referring now to FIG. 1, an embodiment of the overall anti-contaminant system comprises a primary system 110 and one or more apparel and/or accessory subsystems 120 that work together to protect the occupants of and/or personnel accessing a sterile, protected, and/or hazardous area. The primary system and subsystems disperse contaminants, including pathogens, away from the protected area occupied by living organisms susceptible to infection and/or contamination.

The overall system is multi-tiered and integrates apparel, garments, apparel attachments such as straps, pockets, hooks, belt loops, ties, clips, etc., and/or accessories such as eyewear, face masks, forearm guards, leg guards, belts, gloves, etc., to deliver a protective air curtain projected outward from the user's body, and particularly away from the user's eyes, skin, nose, and mouth.

It should be understood that word(s) and terms relating to PRIMARY SYSTM(S), SECONDARY PRIMARY SYSTEM(S), SUBSYSTEMS, WEARABLE SUBSYSTEMS, SYSTEM DEVICES, SUBSYSTEM DEVICES, SYSTEM COMPONENTS etc., may be used herein to assist in understanding the purpose and meaning of the disclosure.

Within the context of this disclosure, the term PRIMARY SYSTEM generally refers to the largest (physically biggest), command and control component of the unified anti-pathogenic system. However, there may be exceptions to this general rule.

A primary system enclosure or secondary primary system enclosure such as a private home, residential complex, medical building, scientific research facility work building, vehicle, bed, bassinet, incubation system, baby carrier system, crib, stroller system, vehicle seat system, playpen, etc., may simultaneously protect one to several persons, animals, or living organisms within a protected area or vicinity.

However, the primary system's protected area or vicinity may, to the extent possible, extend anti-pathogenic protection beyond the designated area.

This extension would occur by the initiation of at least one person donning or removing at least one wearable or portable anti-pathogenic subsystem (e.g., glasses. armguards, gloves, face shields, infant carriers, etc.), within and optionally exiting out of or being outside of and optionally entering into at least the one primary system protective area.

Other than the PRIMARY SYSTEM, all other parts of the collective system are considered SUBSYSTEMS, irrespective of specific word delineations, e.g., subsystem device, subsystem component, secondary primary system, wearable subsystem, etc.

However, upon the occurrence of certain events, (e.g., a break in communication between the primary system and a subsystem, the removal of a primary system component, an artificial intelligence determined event, or another pre-programmed event) a subsystem may become the temporarily controlling primary system, also known herein as the SECONDARY PRIMARY SYSTEM. A secondary primary system will usually be a smaller, non-wearable anti-pathogenic subsystem enclosure, such as a vehicle. When the secondary primary system comes back within a specified range of the primary system, it will generally revert to a subsystem controlled by the primary system, though in certain cases it may be programmed not to.

For example, a Tesla (or other vehicle) may be inside a garage in an anti-pathogenic primary system home. The home is programmed as the controlling primary system and the Tesla is a secondary primary system or subsystem. Every other anti-pathogenic device or system inside the house would also be considered a subsystem (but can also be otherwise programmed). For example, an infant armguard, glasses, baby carrier etc., could all activate simultaneously while inside the house if pathogens or carcinogens are detected by a subsystem either within or outside of the house.

However, if the Tesla leaves the house while the driver is wearing a pair of anti-pathogenic glasses, and a baby is inside the car in an anti-pathogenic car seat carrier, the car would be become the secondary primary system. If the driver's anti-pathogenic glasses or the carrier are exposed to germs or smoke, the car would receive the communication from those subsystems rather than the primary system inside the house. However, the car could still relay the exposure and activation signals to the house's primary system.

When the car returns to the house, it once again becomes one of the subsystems controlled and monitored by the house.

As another example, an anti-pathogenic enclosure such as an infant carrier could also be a primary system designed to protect infants and might link to armguards, wristbands, other carriers and may also link to a house.

Although size is a consideration for determining whether a given subsystem is a secondary primary system, enclosure capability is also important. Secondary primary systems include infant carriers, cribs, and bassinets; medical (and other) beds; vehicles; etc.

Unlike primary system enclosures such as homes, vehicles, baby carriers, bassinets, medical or bariatric beds, etc., wearable subsystem device(s) such as anti-pathogenic glasses, face masks, face shields, armguards, glove systems, wristbands, watch systems, body vests, etc., are primarily designed to be conveniently deployed, individual anti-contaminant subsystem components.

Furthermore, these systems complement (when so programmed) the primary system by extending the anti-contaminant protection afforded by the primary systems to the extent possible, beyond the primary system's area of coverage to ensure that contaminants are not carried back by associated individuals upon leaving and returning to the proximity of protected persons, animals or other living organisms within the primary system or secondary primary system enclosure or the defined area of protection.

Additionally, wearable subsystems and system component extensions may provide early warning notification prior to subsystem reentry (i.e., due to a wearer's exposure) into the primary system enclosure or area.

Communication to a primary system may occur prior to subsystem(s) reentry into the primary system enclosure or proximate to the area protected by the primary system enclosure.

In a preferred embodiment, the primary system controls an area within a protected enclosure but can also be placed, mounted or integral within or outside of the protected area.

Additionally, a primary system does not need to be a single system enclosure and may consist of multiple components, each component system device being substantially programed to authenticate (or parent) at least one subsystem extension.

Body wearable subsystems may be utilized as individual personal protective equipment (PPE). However, these subsystems have the ability to integrate with the primary system and other subsystems but are also designed for use in the therapeutic, industrial, sports, and fashion industries.

Any non-wearable, removable, and/or interchangeable component of a primary system air import/export configuration such as: pressure tubing releases, valves, mass intake mechanisms, thrust cylinders, pylons, openings etc. may be programmed to collectively disengage from the primary system enclosure to form a secondary primary system.

In addition to the aforementioned secondary primary system, any of the subsystem component devices may communicate or alert via any integrated portion of the collective system, including body wearable subsystems devices.

For example, several anti-pathogenic pylons may be integrated into the walls of an infant's baby crib. The integration or attachment of the pylons to the protective bed enclosure forms a single unified anti-pathogenic primary system enclosure.

However, when the pylons are detached and placed at various demarcation points or distance configurations away from or around the proximity of the enclosure (expanding or diminishing anti-pathogenic coverage), the newly deployed pylons or other import/export device configurations may become the secondary (non-wearable) or controlling primary systems for the purpose of managing the contaminant free zone to the safest variable extent possible.

The system provides protection to users both inside and outside the protected enclosure. Protection inside the enclosure is primarily provided by the primary system 110. Protection outside the enclosure is provided by the worn subsystems 120. The primary system and the subsystem(s) on users'apparel, garments, accessories, etc., are in communication (e.g., via internet, WAN, LAN, WLAN, cellular signals including WIFI and Bluetooth networks as well analog, 2G, 3G, 4G, 5G and associated progressive generation wireless network technologies, etc.) so that the primary system can monitor the status and location of the subsystem(s).

The primary system includes a central controller 112, one or more primary devices 114, and one or more optional enclosure access points 116. The central controller 112 comprises one or more computing devices, and is in communication with the primary devices 114, optional enclosure access points 116, and the subsystems 120.

The primary devices 114 comprise one or more mass projection means including fans, turbine systems, pressure release systems, negative and positive currents, etc. The system uses the mass projection means 114 to separate at least one defined area or radius (e.g., the protected enclosure) from another defined area by introducing a mass barrier (such as exported air) to prevent intrusion into the protected area from foreign contaminants, thereby preventing or reducing cross contaminations and infections.

Optional enclosure access points 116 comprise an entry and/or exit device, e.g., a door. The access points are in communication with the central controller 112, such that the central controller 112 may deny entrance to a user that needs to be decontaminated by, e.g., preventing the door from opening.

The mass projection means may be affixed at or integrated within at least one single access point proximate to at least one protective area, and up to 360 degrees vertically and/or horizontally around the top, bottom, sides, or floor of the protective enclosure.

The mass intake mechanism of the primary devices may be affixed to or integrated with an environmentally supplied mass source (e.g., may intake air from the environment) or the system may be self-contained employing an adjustable pressure source. The primary devices may also use one or more active and/or passive purification methods to kill, degrade, neutralize, and/or reduce dangerous or unhealthy airborne contaminants. Some of these purification methods include ionization currents, ozone generators, air purifiers, air filters, electrostatic filters, HEPA Filters, carbon activated filters, sprays, chemicals, UV lights, etc. The primary devices also optionally include a temperature and power output setting.

The mass projection means provide an air force field barrier which defends at least one to several people or living organisms within the protected area who would ordinarily be susceptible to airborne chemical contaminations or infectious dangers such as Covid-19, SARS, Ebola, tuberculosis, influenza, common colds, toxins, allergens, chemical aerosols, spoors, vapors, smoke, or steam. The force field barrier created by the system can also be used to repel germ and pathogen carrying insects such as flies and mosquitos from people, animals, and other living organisms within the protected area.

The primary system protected area may include various types of stationary or mobile platforms such as enclosed bank teller work or transaction barriers, construction trailers, forestry fire and EMS commands stations, incident command stations, heavy equipment enclosures where dust and harmful chemicals are released from building construction and demolition, hospitals and operating rooms, bariatric bed enclosures, bassinets, and law enforcement structures and holding facilities.

The subsystem components 120 may be attached to or integrated with at least one optionally worn piece of protective apparel or garments such as sweaters, jackets, vests, trousers, gloves, mittens, shirts, undergarments, etc., and/or at least one optional worn protective accessory devices such as eyewear, face masks, face shields, wristbands, watches, knee guards, shin guards, arm guards, elbow pads, hats, helmets, boots, etc. Each subsystem component comprises at least one expulsion device. The subsystem components are in communication with the primary system to alert the primary system when a decontamination event has occurred.

The primary and/or the subsystem(s) may intermittently or continually repulse, repel, deflect, diffuse, expel, neutralize, treat, and/or kill airborne contaminants encroaching within the protective area by directing forced, compressed, focused, expanded, flowing, sprayed, misted, liquefied, ionized, filtered, treated, cooled, and/or heated air or other mass matter away from at least one protective area.

In an embodiment, if a subsystem wearer tries to leave the enclosure without first activating any worn subsystems, the primary system can prevent the door from opening until the subsystems have been activated. Furthermore, if the primary system detects a situation where a user may potentially have been exposed to contaminants outside the enclosure (e.g., one of the user's subsystems was deactivated or communication between the subsystem and the primary system was disrupted), the primary system can prohibit the wearer's access to the enclosure until a decontamination process is completed.

To accomplish this, the system further comprises an optional alert that is triggered when the primary system and/or the subsystem(s) detect that either system failed to activate during any pendency period when the systems were apart or removed from the proximity of the other and then returned to the proximity of either system. The alert is for the purpose of initiating preventive decontamination (DECON) procedures prior to allowing re-entry into the protected area and establishing reintegration with the primary system.

FIG. 2 provides a top-down view of an example primary system 200 that is an enclosed work area within a medical research facility or experimentation laboratory that permits trained doctors and scientists to enter and exit hazardous areas within the facility at designated places and times. Within the facility may be a sterile area 210 enclosed by, e.g., steel walls and/or projectile resistant Plexiglas. The sterile area 210 may be accessed by one or more doors 260.

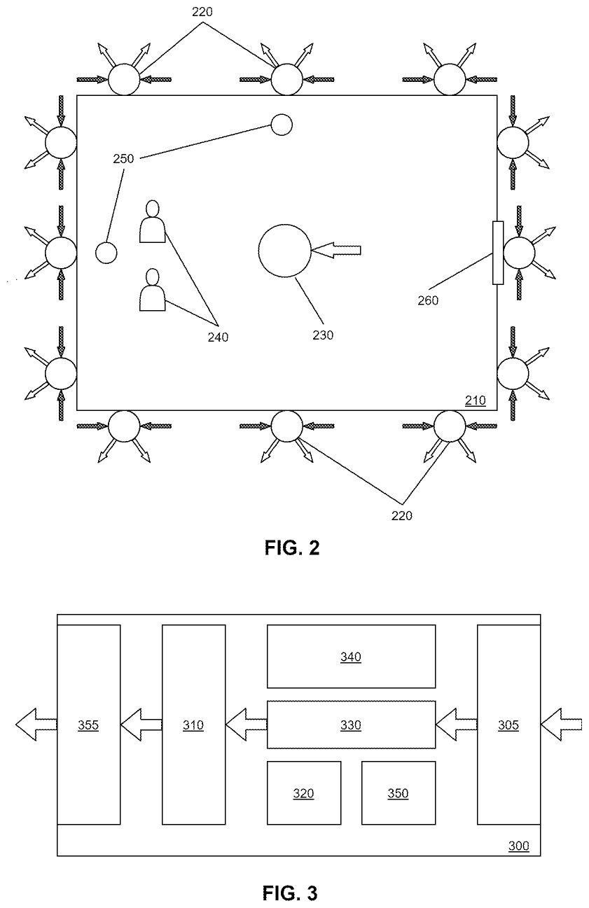

Several large (e.g., 3 meters×6 meters) anti-contaminant primary system devices 220 may line all sides of the enclosure. Each primary device is configured to import air from the environment (represented by the filled arrows) or a self-contained source (e.g., a compressed air source), optionally filter the mass, and export the mass to the environment (represented by the unfilled arrows). Because of the operation of primary devices, air continually flows away from the enclosure. Optionally, the primary devices may be vented to the enclosure. Primary devices are explained in more detail with respect to FIG. 3.

Disposed in the ceiling of the enclosure there may be one or more additional primary devices 230 that are configured to intake air and ionize or treat airborne contaminants that are vacuumed upwards (represented by the unfilled arrow) from the lower part of the enclosure. The primary devices 220 and ceiling primary devices 230 operate together to export contaminants from the protected enclosure. As such, personnel 240 working in the enclosure are protected from disease and harmful chemicals. In an embodiment, and as described further below, the personnel 240 may be wearing one or more subsystem devices to prevent cross-contamination when leaving and reentering the protected area. To keep a steady supply of oxygen for the personnel 240, one or more optional oxygen sources 250 may be provided within the enclosure.

FIG. 3 illustrates an example primary system device 300. As illustrated, each primary system device may comprise a mass intake port 305, mass expulsion means 310, a power source 320, a filtration system 330, a communication module 340, a control module 350, and a mass export port 355.

In an embodiment, a filtration system 330 may employ a barrier which may be externally applied over the mass intake (used synonymously with mass import) area of the thrust cylinder, or it may be internally placed in a configuration designed to cause the ion generator 440 antenna (antenna not shown) to contact, or otherwise facilitate the discharge of ionization or directed energy onto the surface of the filtration barrier 330 or proximate the filtration barrier.

The contact or proximate discharge of energy from the ion generator antenna 440 upon or into the filter barrier, is for the purpose of destroying and/or neutralizing contaminants which may remain on the filter surface after air importation, and to prevent user contamination upon removal, replacement or disassembly of the system and/or component devices,

The mass expulsion means 310 may comprise any type of device for expulsion of mass, e.g., a fan driven by a motor, a blower, a pump, etc. The power source 320 may comprise any suitable source of power (e.g., a rechargeable or non-rechargeable battery) sufficient to power the mass expulsion means. When operating, the mass expulsion means intakes air (or other mass) through the mass intake port 305, passes it through the filtration system 330 to remove contaminants, and expels the air via mass export port 355, as shown by the large arrows.

The filtration system 330 may comprise one or more filtering devices, including, but not limited to, HEPA filters, carbon filters, electrostatic filters, ionizers, ozone generators, air purifiers, or any other applicable type of filter or combination thereof.

The control module 350 is configured to control the operation of the primary device, including the force produced by the mass expulsion means. For example, a fan may have an adjustable fan speed, and the fan speed may be user-adjustable or automatically adjusted based on environmental conditions. The control module is electrically connected to the mass expulsion means 310, the power source 320, the filtration system 330, and the communication module 340 to facilitate control of each module.

The communication module 340 is configured for electrical communication with one or more of: the central controller 112, other primary devices, and subsystem devices. The communication module may include a wired network interface, such as an Ethernet interface or a USB interface, or can include a wireless interface, such as an interface in compliance with an IEEE 802.xx standard, for example including Wi-Fi, Bluetooth, or WiMAX standards, or can include a combination thereof.

Returning to FIG. 2, the primary system devices may be fixed in placed around the periphery of the enclosure, or they may be mobile. The primary system devices may be synced together through operation of their communication modules 340, so they operate in unison to provide a constant pushing of air mass, and thereby airborne contaminants, away from the enclosure.

Personnel 240 coming into the sterile area may be required to wear hazardous material suits because the facility contains high concentrations of toxic chemicals or pathogens such as: cyanide, coronavirus, and/or various strains of influenza, which, if accidently released, could become airborne resulting in injury or death to people within and outside of the facility.

In an embodiment, the hazardous material suits may be equipped with one or more anti-pathogenic subsystem devices. For example, relatively large (e.g., 1 meter×1 meter) anti-pathogenic subsystem devices may be attached to strap and harness fixtures on the back of the PPE hazmat suits.

Additionally, or alternative, the personnel may be wearing other protective devices, such as face shields, goggles, etc. In embodiments, a transparent face shield or pair of goggles may include one or more anti-pathogenic subsystem cylinders attached to or integrated into each side portion of the face shield or goggles.

Protocol within the facility may direct that upon entering or exiting an exposure area outside of the sterile area, a person who is wearing any anti-pathogenic (used synonymously with anti-contaminant) system device must have it continuously on upon entering, exiting, and reentering the controlled enclosure. This procedure may be applicable throughout the facility or to specific areas such as biological or chemical research and testing rooms depending on the potential exposure level to people, animals, and living organisms established by the governing or controlling authority.

Accordingly, the primary system devices and the subsystem devices (e.g., the anti-contaminant cylinders attached to or integrated into hazmat suits, goggles, face shields, etc.) are in communication with the primary central controller 112 that is configured to coordinate the operation of the primary system and subsystem devices.

The central controller 112 monitors the status, including location and operation status (e.g., on or off) of each primary system and subsystem device. The location of subsystem device(s) may be determined using any available location-determining means, e.g., GPS, proximity detectors, etc.

The central controller 112 may also be in communication with entry and exit doors of the sterile area and possibly throughout the facility, and can prevent a door from opening if the subsystem devices of the person requesting the door be opened are not operating.

In an alternative embodiment, each entry and exit door 260 may have a proximity detector that detects nearby subsystem devices, and the door may refuse to open if it detects a subsystem device nearby that is not turned on.

The system may require that the subsystem devices are on continually while outside the sterile area, or require the user to undergo decontamination before entering.

In designated areas the personnel anti-pathogenic subsystem devices may be turned off without causing a disruption in the communication algorithm of the system. However, if personnel attempt to enter a sterile or hazardous area without the worn subsystems being activated, the personnel will be denied entry, and may (depending on protocol) be required to undergo a decontamination procedure before being allowed into the sterile or hazardous area.

In an embodiment, animals, plants, or other living organisms within the controlled environment for experimentation purposes, may be secured in sterile portable carriers fitted with subsystem devices integrated within the enclosure's exposed openings, or secured on about or around the proximity of the enclosure's openings. The subsystem devices may be activated when manually transported from the controlled environment into other parts of the facility.

The subsystem expulsion device(s) can be incorporating into many different accessories, including, e.g., gloves, face masks, wristbands, forearm guards, eyeglasses, watches, etc. The expulsion device(s) may also be incorporated into work uniforms, medical uniforms, hazmat suits, or other items of clothing to additionally protect the wearer.

Primary system devices and subsystem devices may be configured to output a signal that indicates compatibility with the system, such as via a communication protocol, an application protocol, a SaaS application, a unique identifier, etc. This allows inter-compatibility between components designed to be used with the system.

In an embodiment, the expulsion device(s) and/or the accessory and/or clothing incorporating the device may include a motion detector (e.g., an accelerometer). The subsystems may be configured such that triggering the motion detector activates the expulsion device(s), so users can quickly protect themselves, e.g., if someone nearby sneezes. The accelerometer (or other motion detector) will detect the sudden movement and cause the activation of the expulsion device. Alternative, a specific pre-determined movement (e.g., shaking the device two times in rapid succession) may trigger the expulsion device.

In an embodiment, the expulsion device(s) may be remotely controlled by an app on a suitable portable device (e.g., a cell phone, a tablet, etc.) that can detect the sound of someone sneezing nearby and automatically activate the expulsion device(s). The app may also be triggered by a particular motion, e.g., shaking the portable device two times in rapid succession.

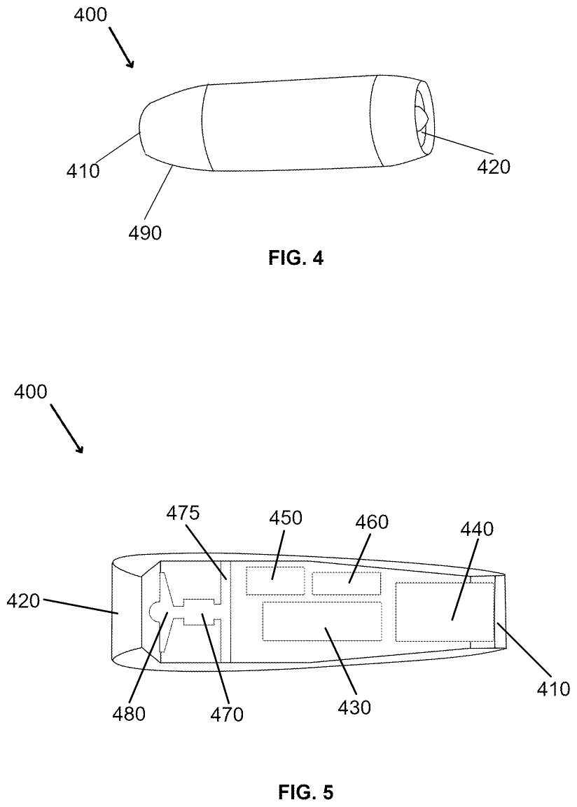

An embodiment of a subsystem expulsion device 400 is illustrated in FIGS. 4 and 5, in the form of a thrust cylinder. FIG. 5 is a cutaway view, showing the internal components of the thrust cylinder. Each thrust cylinder includes an intake opening 410, an export opening 420, a rechargeable battery 430, an ion generator 440, a fan speed controller 450, a communication module 460, a motor 470, a stator 475, and a fan 480. The thrust cylinder may also include several buttons (not shown), such as a power button, a fan speed button, and (optionally) an ionization button. Further, the thrust cylinder may include an internal filter, such as a HEPA filter and/or a carbon fiber filter (not shown) and a charging port (not shown) for charging the rechargeable battery 430.

The motor 470 may comprise a brushless, brushed, linear, servo, or stepper motor, and drives the fan 480 based on the fan speed set by the fan speed controller 450. The fan speed controller 450 may be user-controlled, e.g., via a fan speed button or switch. The ion generator 440 may be configured to generate negative ions when power is supplied, and may be controlled by a user-accessible switch or button.

The communication module 460 operates to electrically communicate with one or more of: the central controller 112, the primary devices, and other subsystem devices. The communication module may include a wired network interface, such as an Ethernet interface or a USB interface, or can include a wireless interface, such as an interface in compliance with an IEEE 802.xx standard, for example including Wi-Fi, Bluetooth, or WiMAX standards, or can include a combination thereof.

In an embodiment, the rechargeable battery may be accessed by twisting and removing one end, e.g., the intake ends 490. The filter may also be replaceable by a similar process.

In operation, the motor 470 drives the fan 480, causing the intake opening 410 to import or take in air mass and contaminants, pass the air mass through the filter and ionizer 440 to remove contaminants, and expel the air through the export opening 420, thus thrusting or exporting air mass and contaminants away from a person or area.

If present, the ionization button may be configured to activate the ionizer 440, thus discharging negative particles into the air to eliminate airborne germs and contaminants. An additional option for the thrust cylinder is a button that activates a UV light that kills airborne pathogens.

In an embodiment, the export opening 420 may be adjustably focused (like a pair of binoculars) to push mass volume forward of the wearer's body to repel airborne contaminants at various distances. This adjustment would cause contaminant expulsion at narrower or wider demarcation points forward of or away from the wearer.

The thrust cylinders may be elongated or oval shaped, e.g., in a specific embodiment, they may be shaped like jet engines. They may be made of any suitable light-weight material, e.g., titanium, aluminum, steel, carbon fiber, etc.

In addition to their use for protection from contaminated air, thrust cylinders can be used for other purposes, such as cooling, clearing smoke, blowing away insects, etc. In an embodiment, a thrust cylinder may include an air-cooling element with user-adjustable temperature settings.

In an embodiment, instead of having an air intake opening, a thrust cylinder may be connected to a compressed air source that may be worn on the user's body, e.g., an air tank.

In an embodiment, a thrust cylinder may include an accelerometer or other device configured to detect motion. The accelerometer may be directly or indirectly electrically connected to the fan motor, so that when a sudden motion is made, the fan is activated. As such, the wearer of the thrust cylinder may quickly react to a contamination event (e.g., someone nearby sneezes) by quickly moving the subsystem device, and the fan will activate without the power button being pressed. In some embodiments, the motion that needs to be made may be user-selectable, e.g., a specific number of shakes, etc.

The activation of the system may also be autonomously responsive to a set of instructions implemented by a machine learning program and/or a set of instructions such as an FPGA and/or ASIC assisted learning implementation chip, a computer program, an AI implemented learning program or other communication means known in the prior art.

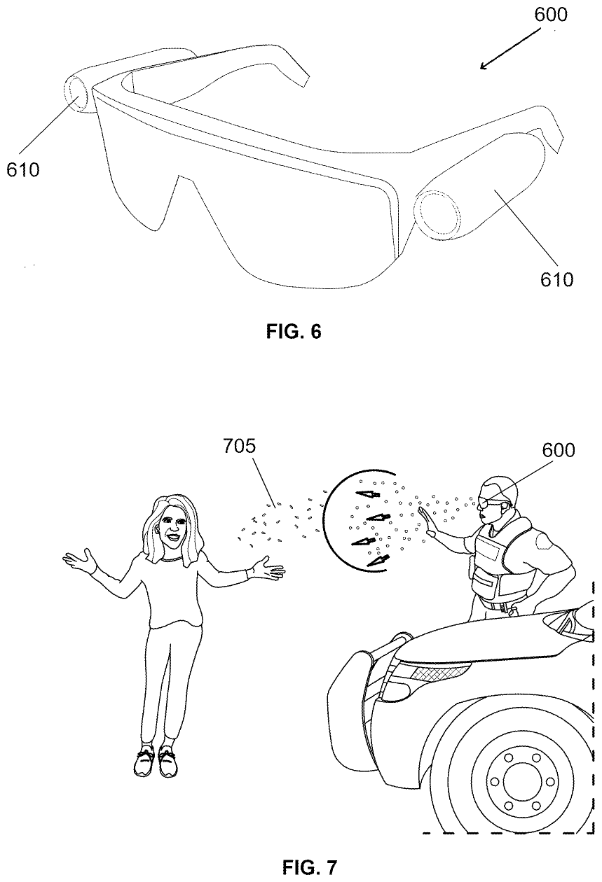

In a particular embodiment illustrated in FIG. 6, a pair of eyeglasses 600 includes two detachable air mass thrust cylinders 610. Alternatively, the thrust cylinders may be integrated into the frames of the glasses. The thrust cylinders are positioned so that the export openings are forward of the lenses of the glasses, to prevent the exported air mass from interacting with the lenses, e.g., causing them to fog. Attachable thrust cylinders would include a receptacle that can snap onto or slide over eyeglass arms. In either embodiment, the air mass thrust cylinders may be rotatable to allow the user to alter or control the direction of the airflow.

FIG. 7 illustrates the eyeglasses 600 in use to protect a law enforcement officer from airborne pathogens 705 when interacting with a member of the public. When the officer activates the eyeglasses, the force of the expelled air (illustrated by arrows) pushes the airborne pathogens away, protecting the officer.

Thrust cylinders for use with eyewear may also include a filter screen placed over the intake opening to prevent foreign objects, e.g., bugs, hair, etc., from entering the cylinder.

In additional embodiments, thrust cylinders may be integrated into or attached to other face wear and face protection apparel, such as masks. On masks, the thrust cylinders may be attached proximate the jawline on either side, or a single cylinder may be attached under the chin.

In a particular mask embodiment, the mask may comprise multiple layers of material. Mass export devices may be configured such that, after importing, and optionally filtering and/or ionizing any imported contaminant, the air mass is then displaced throughout the periphery and between the bottom and top layered material of the mask where it is then exported though the outermost layer of the mask surface material via hundreds of small vent openings covering the front area of the mouth and nose cavity, as well as the entire multidirectional cross-section surface areas of the upper and lower jawline.

Mass export subsystems may also be integrated into a device that protects both the eyes and mouth, e.g., a firefighter mask. In this embodiment, mass can be projected from one or more vents and/or integrated with a mask attachment system (e.g., goggles, eye glasses, firemen's shield and mask systems, etc.). This pushes smoke and fire outwards and away from the firefighter's eyes, so he can see and rescue trapped people and animals in smoke-and fire-filled homes and structures. High pressure subsystem devices can also attach to the firefighter's clothing, or hung around his body, to push smoke and fire away.

With respect to the mask portion of a firefighter mask (e.g., the part that protects the mouth), one or more small mask export vents may be integral with or affixed to the surface area of the mask to force matter sideways over at least one cross-sectional surface area of the mask portion of the system.

This operates to form vertical and/or horizontal mass force-field barriers above the surface area of the mask and prevent airborne contaminants from contacting or adhering to the mask surface material to prevent cross contamination by a user's hands or other body parts on the material surface.

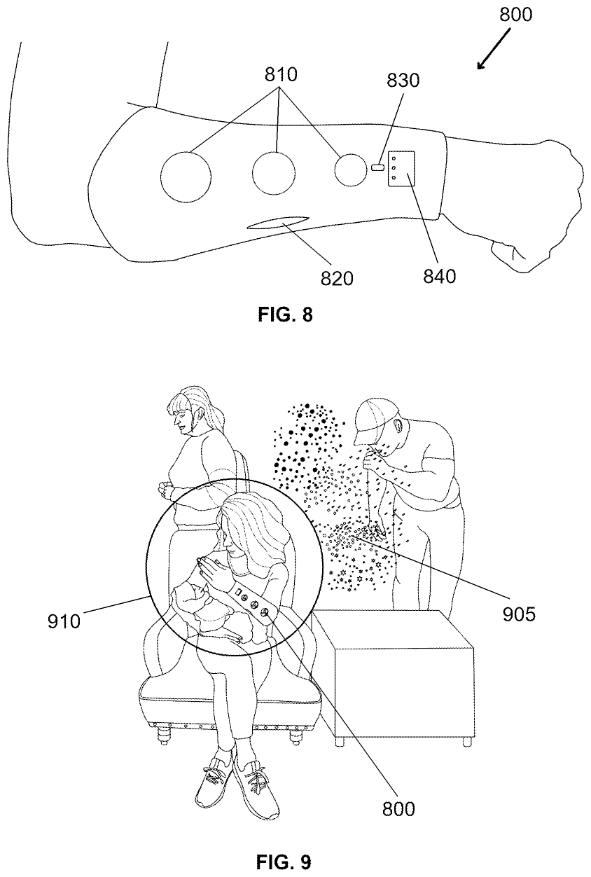

FIG. 8 illustrates an embodiment of an armguard 800 with one or more mass export vents 810. The armguard also includes an intake vent 820, a power button 830, and a display panel 840 including a fan speed button, an ionization button, and a sync button. One or more mass export devices, which function similarly as those described with respect to FIG. 4, are integrated within the armguard. The mass export devices intake air through the intake vent 820, filter the air with an internal filter and/or ionizer, and expel the filtered air out the export vents 810. In another embodiment (not shown) an armguard (e.g., for use by a mother in protecting an infant) may be collapsible, sectioned, or partitioned, so as to fit inside a mother's infant necessity bag for convenient carry.

The buttons function as described with respect to FIG. 4. The sync button allows the armguard to sync with a portable device such as a cellphone (e.g., using Bluetooth). When synced with the armguard, the cellphone may be used to control settings of the armguard, e.g., through a downloadable cellphone app. Also, a setting may allow for motion of the cellphone (e.g., detected by an accelerometer) to signal the armguard device to activate. The display panel is configured to display the status of various settings, e.g., fan speed, etc., to the user.

Any of the other primary systems and/or subsystems disclosed herein may be synced with a cellphone (or other portable device) in like manner.

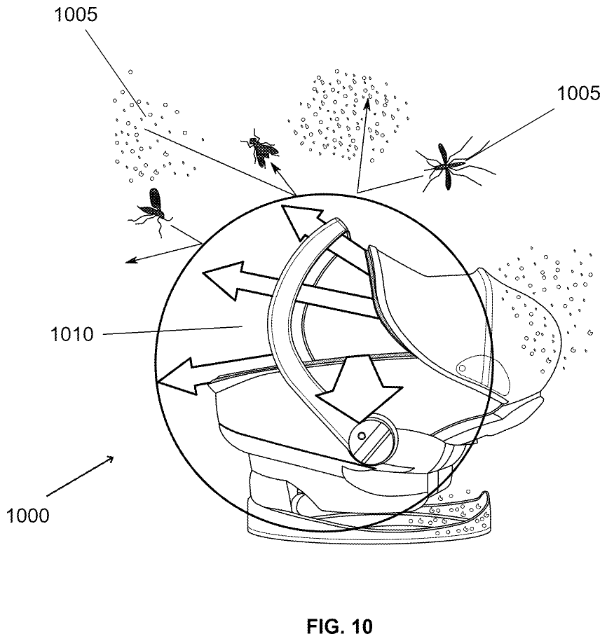

FIG. 9 illustrates the armguard 800 in use to protect an infant held by a caregiver. After a sneeze projects pathogens 905 into the air, the caregiver activates the mass export devices on the armguard. When the armguard's mass export feature is activated, the anti-contaminant function may be facilitated and enhanced by any combination of circular, waving, or cross-sectional motions of the wearer's arm over, about, or around the infant, thereby creating a multi-directional air force field shield 910 around the infant. As such, the manual motion by the wearer assists in pushing away airborne pathogens and contaminants descending down or projected towards an infant.

In another example, a glove may be equipped with a bracelet-like fitting that rotates around the circumference of the wrist. The wrist fitting or wristband includes a snap-in anchor that is configured to receive a thrust cylinder.

The glove may be vented with a hollow hard material surface. The surface contours and follows the outside shape of the user's palm and allows the thrust cylinder(s) to align with a hollow plastic opening under the palm so that air mass that is exported from the device is channeled under the palm and out through the palm vents. This allows the user to open his or her hand wide and expel air mass from the palm area of the hand, causing filtered air mass to push smoke and irritants away from the user's body.

Another embodiment, illustrated in FIGS. 10 through 13, uses multiple anti-pathogenic air-force-field system devices on an infant carrier 1000 to protect infants from disease. In a particular embodiment, several devices are positioned at strategic places around the infant carrier to blow away (as indicated by the arrows) pathogens and other contaminants 1005 as shown in FIG. 10. Infant carriers and enclosures may also be size adjustable to accommodate for rapid growth of infants and toddlers.

Infant carriers generally include at least one opening 1010 through which the infant may be retriever, and the devices may be place around the opening, e.g., proximate the periphery of the opening. In this embodiment, the devices primarily protect the opening of the infant carrier from pathogens and other contaminants.

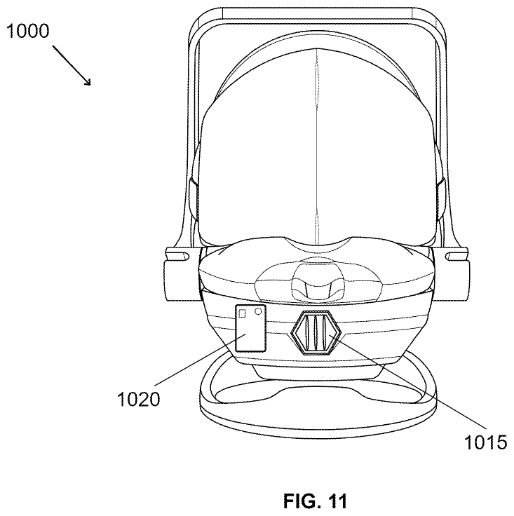

As shown in FIG. 11, the infant carrier 1000 also includes an intake port 1015 and a control/display panel 1020, which function substantially as described herein with respect to the armguard embodiment.

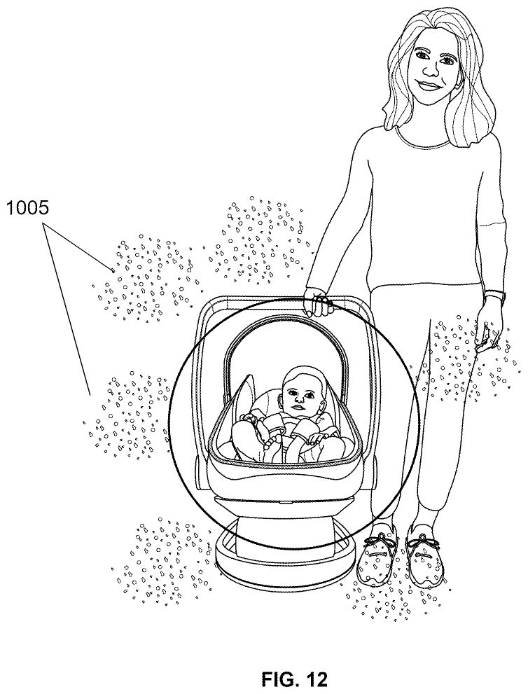

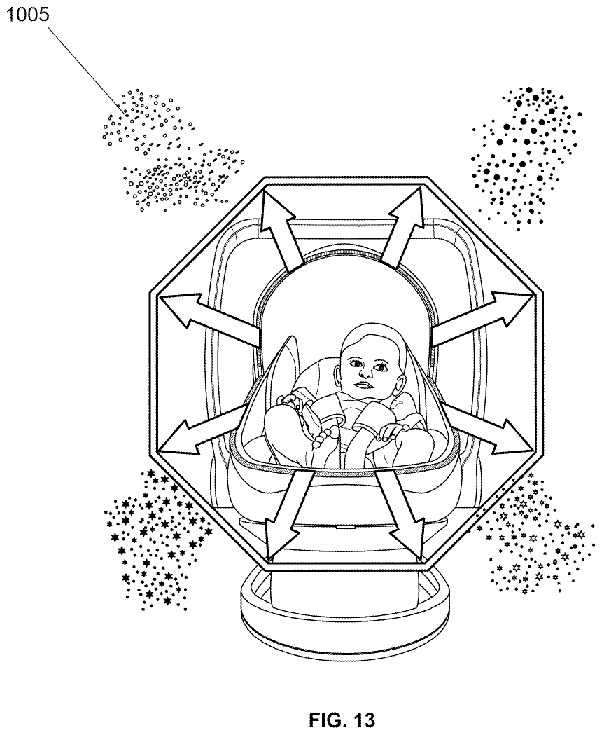

In another embodiment of an anti-pathogenic infant carrier, the anti-pathogenic devices may be placed around the carrier such that they provide 360 degrees of protection from contaminants 1005 around the infant carrier, as shown in FIGS. 12 and 13.

While an infant carrier is illustrated, the same principles may be applied to other infant containment devices, such as baby carriages, cribs, etc. Furthermore, the same principles may be applied to various animal carriers, e.g., dog and cat crates, bird cages, small animal cages, etc.

In any of these infant or animal carrier embodiments, the primary system devices may be in communication with one or more worn subsystem devices (e.g., worn by the parent or caretaker), such that when any of the subsystem devices activates, a signal is sent to the primary devices, and the primary devices activate in response to this signal. This allows the parent or caregiver to easily activate the primary devices (i.e., the devices attached to or integrate with the infant carrier) by causing their worn subsystems (e.g., on an armguard) to activate. As explained herein, the subsystems may be activated by pressing a power button or, if the subsystem is equipped with a motion detector, by quickly moving the subsystem.

Furthermore, in the embodiment where the subsystem devices are triggered by a signal from a portable device (e.g., a cellphone), the initial trigger event may cause a chain reaction of activating devices, e.g., the parent's cellphone may detect a nearby sneeze, and in response send a signal to the parent's armguard subsystem. This causes the parent's armguard to activate its anti-contaminant devices and also send a signal to the primary devices on the infant carrier, causing them to activate also.

Any of the disclosed subsystem devices may include a system setting so that users can limit power usage by disengaging or turning off the ionization function of the device and allowing the system to push contaminants away while only using the fan function.

Further, any subsystem embodiment, including eyewear, masks, face shields, armguards, gloves, may include elements for adjusting the direction and/or force of the exported mass. For example, export cylinders may be rotated (e.g., at their attachment point), and vent openings may include one or more slats that may be adjusted to control the direction of flow of the air (or other mass). To adjust the force, export cylinders may include a focusing ring, and other embodiments may include a toggle, switch, etc., to control the speed of the fan (or other expulsion means).

Another example of an anti-pathogenic primary system is a school classroom. In this embodiment, there are several primary system devices in the ceiling that are designed to extract contaminants (e.g., pathogens) up and away from the children in class. There are also several strategically angled vent openings on the floor and on the baseboards.

As the system operates, air is pushed up and over the floor from the baseboard vents and intercepts the air being pushed up from the floor vents. Because the two air masses meet at a slight upward angle, a force field blanket of protective air forms above the floor of the classroom. The air is then forced upwards, pushing dust and airborne pathogens and contaminants upwards; these contaminants are then sucked up into the vents of the ceiling.

Another example of a primary system anti-contaminant enclosure is a transportable and/or convertible tactical public safety mobile or ICS (Incident Command) headquarter vehicle such as: crime scene investigation trailers, buses, patrol vehicles, SWAT vehicles or other initial response police and/or logistical law enforcement support related vehicles.

Large scale mobile anti-contaminant ICS vehicles may also include forestry fighter trucks and rescue vehicles as well as urban fire truck and rescue ladder bucket vehicles, which may also be designated as initial response short-or long-term command-and-control or incident command headquarter vehicles.

The air quality environments within these stationary and mobile command and control enclosures are often mandated by local, state, and/or federal laws and regulatory boards or commissions such as the EPA and OSHA as well as state and county departments of health and safety established to ensure environmentally safe compliance to prevent mass population infections or contamination outbreaks.

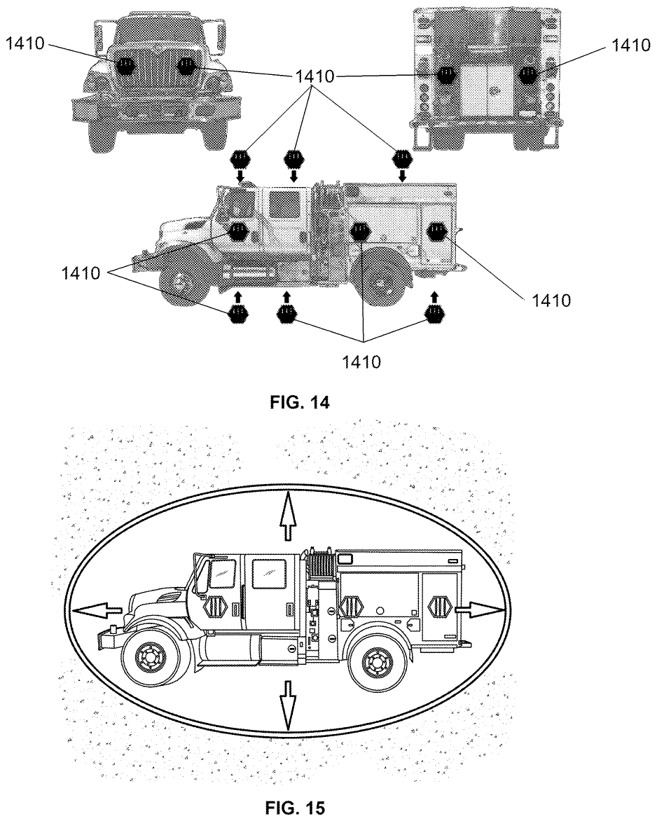

As shown in FIGS. 14 and 15, a firetruck for example may be outfitted with several anti-pathogenic system device vents 1410 covering the top, bottom, and side areas of the truck and able to deliver a directionally-controlled air curtain of protection of up to 360 degrees around, above, and beneath the fire vehicle. The system could be used for escaping smoke and fire danger zones in cases where the truck becomes trapped or stalled in heavy smoke or within an actual fire.

Because of the extreme nature of heavy smoke in forest fires that may overwhelm the system's filtration system, the primary air mass intake/export devices are connected to pressurized mass sources, which are self-contained within the truck. However, the functionality of the primary devices could be manual or system controlled to disengage from the self-contained mass intake source and instead use environmentally available mass in lighter smoke or fire situations. The primary system is still effective using only a fan application from the devices, by simply blowing excesses light smoke or fire away from the proximity of the fire rescue vehicle.

As a more specific example, a rescue truck may be equipped with a 360-degree anti-carcinogen air-force-field-smoke system. At maximum PSI the truck can push out air for about 8 minutes of continuous 360-degree air force field coverage. The truck may be divided into zones—e.g., a front vent export system below the front windshield, a crew cabin vent export system below the side cab windows, a top crew cabin vent export system directly over the driver cabin roof, and a rear vent export system. The user can activate each zone separately from the other zones, e.g., to blow smoke away from the engine, only the front vent export system may need to be activated, while to clear the entire front of the truck of smoke to protect the inhabitants from smoke inhalation, the front vent, crew cabin vent, and top crew cabin vent export systems may be activated. The PSI is user-controllable to allow for use in a range of conditions, e.g., light smoke, heavy smoke, etc.

This embodiment may employ wearable subsystem devices as extensions beyond the primary protective area for the purpose of preventing cross-contamination upon reentering the truck.

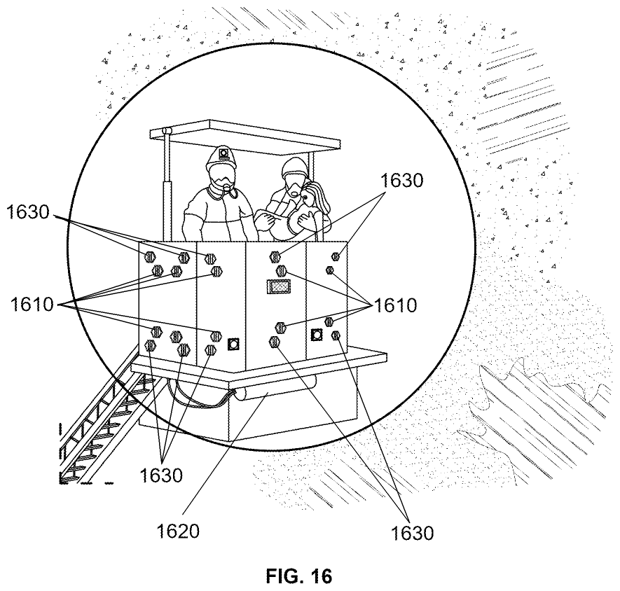

Another example, illustrated in FIG. 16, is a fire truck ladder bucket equipped with a primary system. The bucket comprises a platform and overhead mount that can hold up to three people, who would be insulated within a protectively enclosed 360-degee force field of air upon activation of the primary system.

Several primary system devices 1610 are attached to and around the platform and are configured to expel smoke and fire away from the firemen inside of the bucket as it is lifted into or away from the fire and/or smoke zone. The force is supplied by a tank system 1620 under the platform.

The fire bucket system may optionally include several devices 1630 that export a controlled water supply from a hose system. The water export devices can be configured to work in conjunction with the air export devices.

### PRACTICAL EMBODIMENTS

### Embodiment 1—Sports and Leisure (SUBSYSTEM)

In an embodiment a Yoga instructor requires class participants to wear filtered or ionized air-force-field wristbands, instead of (or in addition to) masks, to push back or wave away lingering germs or “bad air” to prevent or reduce outbreaks such as Covid-19 and influenza while exercising or meditating in class. One student had not purchased any wristbands. However, the instructor permits him to go home and get his air-force-field chest device that is attached to his industrial work utility uniform that he wears at the power plant. He returns to class and activates the device which he then uses to push and wave away the “bad air” from around his body space.

### Embodiment 2—Crowds & Sick People (SUBSYSTEM)

In an embodiment A stands in line inside a bank. A is wearing one air-force-field wristband on his left wrist and one air-force-field watch on his right wrist. B, who is two places behind A, suddenly and violently sneezes. A is aware that airborne contaminants can remain suspended in the air for several minutes and so to protect himself A immediately raises both arms and begins to wave and push the “bad air” away from his body. Because A's wristband and watch are equipped with accelerometers, the sudden movement of A's arms instantly activates both expulsion devices.

### Embodiment 3—Device Syncing & General Use (SUBSYSTEM)

In a continuing embodiment, C, who is standing at the end of the line, is wearing one air-force-field forearm guard. However, C's device is not equipped with a motion detector, but it is synced with his Android phone that has a voice and sound recognition app that recognizes the sound of B's sneeze. As a result, C's forearm guard is automatically activated and so C also waves and pushes away the airborne pathogens from his body in the same manner.

### Embodiment 4—Maternity & Infant Coddling (SUBSYSTEM)

In a continuing embodiment, D, who had been sitting at a desk waiting for the bank manager, is holding her newborn infant wrapped in a blanket in her arms. D had recently purchased an air-force-field armguard for infants to protect her newborn from germs because D was aware infants have weak immune systems. D is about 15 feet away when she hears B violently sneeze and so she immediately starts making circular movements around her infant's face and body to repel any airborne germs. To be extra safe D also stands up and walks towards the exit so that any lingering germs could dissipate before she returns.

### Embodiment 5—Maternity Baby Strollers & Carriers (PRIMARY SYSTEM Stationary/SUBSYSTEM in Motion)

In a continuing embodiment, as D exits the bank, she holds the door open for E who is pushing her infant in a stroller. Inside E's baby stroller is a removable baby carrier. The carrier is fitted with four removable after-market anti-pathogenic infant air-force-field system devices. The devices were strategically fitted to four points around the carrier opening to protect E's newborn.

As E enters into the bank, several thousand microscopic influenza pathogens floating in the air begin to descend towards E's newborn. However, because E had activated the system, as the descending germs close in on the opening of the carrier, they are immediately deflected by the air-force-field protecting E's infant.

### Embodiment 6—Maternity & Medically Prescribed Baby Strollers & Carriers (PRIMARY SYSTEM)

In a continuing embodiment, as D exits the bank, she holds the door open for E who is pushing her infant in a stroller. Inside E's baby stroller is a removable baby carrier. A factory installed anti-pathogenic Infant Force Air-Force-Field System has been installed into the opening around the carrier. The carrier has four air thrust port openings, which were strategically fitted into the carrier frame around the opening to protect E's newborn. E's particular carrier was prescribed by her pediatrician and was covered by her medical insurance because her infant was born with a lung defect.

As E enters the bank, thousands of microscopic influenza pathogen particles floating in the air begin to descend towards E's newborn. However, because E had activated the system, as the descending germs make it close to the opening of the carrier, they are immediately deflected away by the force field of air protecting E's infant.

### Embodiment 7—Work Utility Areas (Primary System & Subsystem INTEGRATION)

In a continuing embodiment, F walks up to the bank window immediately after B sneezed. However, once F arrives at the counter, F notices that air is blowing on him from several fixed positions on the counter and from the glass barrier. F realizes that the bank's primary system anti-contaminant application was activated in response to B's violent sneeze.

F also notices that all of the bank staff members behind the glass barrier are wearing anti-pathogenic devices on various parts of their bodies and that each device has a small green light which meant that they had been activated. F sees one of the bank tellers walk out from the behind the glass and step outside for a few minutes. However, the green light on the teller's anti-pathogenic chest device was not on when he left.

When the employee walks back into the bank and tries to open the door, it will not open and an alarm sound. The bank manager walks over to the teller and tells him that the primary system had alerted because he (the teller) had forgotten to turn on his device before exiting and then trying to reenter into the protective area. The manager tells the teller to go to the bathroom and decontaminate and when he returns, he (the manager) will reintegrate the teller's subsystem device with the bank's primary system enclosure so that he may safely enter back into the work area.

### Embodiment 8—Law Enforcement Public Safety & Insect Bites (SUBSYSTEM)

A is a police officer who is investigating a drug overdose where B was found unconscious next to a dumpster in an ally. C is B's girlfriend and admits that she and B were shooting up heroin just before B overdosed. C also admits to officer A that she has active Tuberculosis (TB). C tells A it may be airborne but she wanted A to know because she was also once a police officer and she cares for officer A's safety.

C stands 10 feet away from officer A and starts to explain what occurred. A thanks her and presses the maximum thrust button on his new slightly tinted industrial strength anti-pathogen glasses. The glasses have two detachable air mass thrust cylinders and were funded by a federal grant and issued to all patrol officers in A's police department because of a recent spike in the number of police officers and EMS personnel in the Metro Phoenix Area who had tested positive for Covid-19 and TB.

### Embodiment 9—EMT & Rescue Personnel (SUBSYSTEM)

In a continuing embodiment A noticed that the two paramedics who arrived in the ambulance were also wearing anti-pathogenic glasses when they transported B to Good Samaritan Hospital. However, unlike A's glasses, the air mass thrust cylinders were integrated into the frames of their glasses. Officer A goes to the hospital as part of his investigation and is eventually able to speak to D who is the emergency room physician who had treated B.

### Embodiment 10—Hospitals & Medical Staff (PRIMARY SYSTEM & SUBSYSTEM INTEGRATION)

In a continuing embodiment A notice that doctor D is in a full biological medical suit and also has a glass shield covering his face. A also notices there are several anti-pathogen devices attached to doctor D's medical suit which cover his head, chest, legs, and back. A asks doctor D if B has died and D told him that although it was close B survived and will be admitted to a drug treatment facility.

### Embodiment 11—Forest Fire Rescue and Incident Command (PRIMARY SYSTEM)

A massive forest fire has been burning in the Tonto National Forest for over two weeks. Fire jumpers team A radios to fire engine command truck B that they are behind the fire lines and trapped on the main hill by fire and smoke. Fire jumpers team A advise they are being consumed by heavy white smoke and they are having difficulties breathing and are in desperate need of oxygen. Team A tells command B they cannot last much longer and may succumb to smoke inhalation. Team A firemen also asks command B to tell their families they love them and goodbye.

The command B incident commander looks on his GPS map and asks A if they can walk 1,000 yards to the bottom of the mountain where there is a fork in the road. Fire jumpers A look through their binoculars and reply that they know where the fork in the road is but they cannot chance it because the fork is the main source of white smoke that they have been trying to avoid.

Command B tells A the fork in the road is the only way to make a rescue attempt. B also says he is sending three additional fire jumpers in fire rescue truck C. He also tells A that rescue truck C is equipped with a 360-degree Anti-Carcinogen Air-Force-Field-Smoke System. B says that according to his GPS truck C can reach them in 15 minutes. A says they are moving towards the smoke now and they should make it there in about 12-15 minutes but they will probably be unconscious or dead if rescue truck C is not on time.

Fire rescue truck C has a three-man crew. The truck captain tells his crew that at maximum PSI truck C can push out air for about 8 minutes of continuous 360-degree air force field coverage. He also tells his crew they will conserve the air pressure and will only use it if necessary because they are all out of portable oxygen (note-fire jumpers are not usually equipped with oxygen tanks).

Truck C and the rescue crew drive towards the fork in the road to rescue fire jumpers A. However, when they are about three minutes from the fork in the road a very thick wall of white smoke emerges and begins to engulf fire truck C. The captain, who is driving truck C, can't see out the front or side windows of truck C.

To compensate, the captain only turns on the front vent export system (below the front windshield), the two-side crew cabin vent export systems (below the side cab windows), and the top crew cabin vent export system (directly over the driver cabin roof), which form a partial air-force-field smoke barrier around the crew cab portion of the truck C.

The captain also sets the PSI to 25% to conserve air that may be needed to rescue fire jumpers A. After the system is activated, there is more visibility and rescue truck C makes it to the rendezvous point. Truck captain C is unable to reach fire jumpers A on the radio and they are not able to see well due to the smoke. However, because some of the smoke has been displaced by the thrust of the system one crew member spots fire jumpers A who are both lying on the ground and appear to be unconscious. The two crew members exit truck C and are able to lift the two unconscious firemen aboard rescue truck C.

As rescue truck C travels back down the mountainside a very heavy cloud of white smoke begins to cover the truck. This smoke is about three times as thick as the first blanket of smoke and the GPS shows that rescue truck C is still five minutes away from command post B.

Captain C is driving and cannot see anything in front of the truck. Due to the heavy smoke, rescue truck C starts to stall and smoke begins to enter into the cab. Truck captain C radios command station B and tells them they are in extreme danger and they are now activating their 360-degree Anti-Carcinogen Air-Force-Field Smoke System at maximum thrust.

Once rescue truck C is activated at 100% PSI a 360-degree force field of air engulfs and protects the entire truck, causing the smoke to be pushed away from rescue truck C, forming a clear sphere of clean air around, on top, and beneath the truck. Rescue truck C is able to make it to the command area B and all of the fire heroes survive.

### Embodiment 12—Mask (SUBSYSTEM)

A is wearing a mask to prevent or reduce his chances of contracting airborne pathogens such as Covid-19, influenza, and the common cold. A has the new style black face mask which has filtered vents and a new face shield design. There are also two detachable anti-pathogenic devices, one on each side of the mask attached proximate to the jawline.

A removes a second mask from his carry bag. The second mask is similar to the first mask, but does not have a face shield attachment. The second mask also does not have the two detachable anti-pathogenic import/export filter devices attached to the sides of the mask. Although it has the same standard filter vents as the first mask, the second mask is only equipped with a single anti-pathogenic mass import device opening under the chin portion of the jaw.

A, who has been talking with B about the recent Covid-19 epidemic, asks B which mask B wants to see. B likes A's first new mask design that A is wearing and asks him to explain how it works. A removes the mask and detaches the face shield from the breathing apparatus.

A then points to a charging port on top of each of the two import/export air mass cylinders that house the batteries and are designed to look like sleek jet engines. At the smaller end of the cylinders are openings that intake or import air mass and contaminants, while the larger ends are designed for thrusting or exporting air mass and contaminants away from a person or area. There is also a power on/off button next to a fan speed button and ionization button.

A twists the import cylinder and removes a small rechargeable battery. A then points to the cool-looking export openings which look like jet turbines. However, A explains to B that unlike a jet engine, the larger export opening is designed to thrust or emit air mass or treatments from the system and not intake air.

B points to a small button next to a blue light on the device near the power button and asks A what it is for. A tells B it is a small ionizer and that it is designed to discharge negative particles into the air that kill airborne germs and contaminants. A tells B that he upgraded his mask to have this feature and there are several methods used in the device to kill germs including a model which uses UV (Ultraviolet) light.

B points to what appears to be a small piece of dirty material inside a clear window on the housing of the device near the air mass intake opening and asks A what it is. A uses a tiny screwdriver to fully open the device. A then uses a pair of tweezers and pulls the material out and tells B that that it is a treated filter. A then replaces it with a new filter and discards the old filter.

A puts the mask back together and places it on his face but he does not reattach the face shield. A tells B that he (A) is a heart and lung surgeon and that he usually only wears the face shield when he is in the emergency room or the operating room at the hospital. He also explains that his specific system is linked to his hospital operating room primary system to ensure germs are not taken into the sterile operating room environment as physicians and medical staff enter and leave during prolonged surgeries.

B happens to be a serial smoker and rather rudely lights up a cigarette in front of A. However, A's cell phone is equipped with an accelerometer (motion detector) that is synced with A's mask device. As a physician A knows that second-hand smoke can cause cancer.

As the smoke from B's cigarette begins to move in A's direction, A shakes his cell phone two times. This activates A's anti-pathogenic mask causing the smoke to blow away from A and back towards the direction of B. A then twist the export cylinders on each device to focus a wider cone of air protection from B's cigarette.

### Embodiment 13—Anti-Smoking Forearm Guard (SUBSYSTEM)

A is inside a Las Vegas casino for a weekend getaway playing various games and slot machines. Although A likes Vegas, she does not like cigarette and cigar smoke. Unfortunately for A, smoking is permitted on the casino floors. A's eyes begin to water and burn because of all of the smoke and so A decides to leave. However, before A leaves, she stops at the bar to have a drink and wait for her friends.

Once A sits down at the bar two women, B and C, sit on the left side of A and begin smoking cigarettes. A has a two-month-old infant at home. Because of the recent increase in Covid-19 cases, when A left the hospital after giving birth her insurance covered an anti-pathogenic infant forearm guard. A uses the forearm guard to shield and coddle her infant around other people, especially during flu and cold season.

A has also been using the forearm guard to cover her own face and push away the “bad air” when she is in crowded places. As the smoke from B and C's cigarettes begins to cover A, she reaches into her handbag and removes the forearm guard and places it on her left forearm.

Because A's forearm guard is equipped with a motion detector (accelerometer), A shakes her arm two times and the device activates. A then places her left elbow upright on the bar causing air from the ports to shield A by pushing the harmful smoke away from A and back towards smokers B and C.

### Embodiment 14—Anti-Smoking Prescription Eyewear Use (SUBSYSTEM)

A lives in New York City and rides the train to work every day. A has bad eyes which also burn because they are sensitive to smoke and chemicals. Because of A's condition, her ophthalmologist wrote her a prescription for a pair of anti-pathogenic air-force-field glasses.

The glasses have two detachable silver light weight titanium cylinders, one on each side of the frame. Each of the cylinder housings is elongated and oval shaped and encloses a rechargeable battery. The cylinders also contain a filter, an import intake opening to receive air, a small internal ionizer, a fan system, and a mass export opening to push out a force-field of air to assist in keeping airborne germs and carcinogens away from A's eyes and respiratory tract.

While A is standing on the train, passenger B, who is standing about five feet from A, starts smoking a marijuana cigarette. A was already wearing her clear prescription glasses. She touches a small button on each cylinder which activates the system. The force of the air from her glasses pushes the smoke away and back towards marijuana smoker B. Several passengers on the train who are also wearing or carrying various styles of anti-pathogenic glasses also activate theirs to keep the smoke away from their faces.

### Embodiment 15—Fashion and Leisure (SUBSYSTEM)

C is also standing on the New York subway train where B has been openly smoking a marijuana cigarette. C sees several passengers putting on their anti-pathogenic glasses to keep from breathing in carcinogens from B's marijuana cigarette. C also notices that the passengers who are wearing glasses mostly have the styles that have detachable elongated oval thrust cylinders.

Because C prefers the finer things in life, she removes a custom pair of anti-pathogenic sunglasses from her designer bag and places them on her face. C's sunglasses are also equipped with an internal ionizer to help kill germs. Unlike the glasses worn by most of the passengers, the cylinders on each side of C's glasses are not detachable and are integrated within the frame. Additionally, C's frames are gun metal gray and made with light weight titanium alloy cylinders with gold trim and are fashioned to resemble two aerodynamic jet engines.

### Embodiment 16—Heat Stroke Treatment & Body Cooling (SUBSYSTEM)

A is a nurse and is inside the gym having a workout on the treadmill. After 30 minutes of running A steps off the treadmill to rest and cool off. The gym's cooling system has recently broken and the temperature inside is about 85 degrees.

A wipes the sweat off her body with a towel but she is still hot. A is wearing a new anti-pathogenic wristband on her right wrist which she received as a Christmas present. This particular model has several upgrades, including an ionizer and an air-conditioning cooling system.

A sits in a chair and turns the cooling mechanism and fan on her wristband to maximum. A then starts using it to blow cool air over her face and body. After several minutes A looks over and sees elderly woman B who has just stepped off the treadmill.

A notices B is stumbling and appears to be in distress. A walk over and asks B if she needs help. B struggles to talk and A immediately recognizes that B is suffering a stroke. The woman passes out but A catches her and gently places her on the floor and beings to render medical assistance. Several people come over to assist A and someone calls 911.

As A renders aid, general manager C asks A how he could be of assistance. A tells C that stroke victims have high core body temperatures and it was imperative to keep B's head cool. A removes her wristband and tells C to put it on and keep it held over B's head. C does what he was asked until paramedics arrived and transport B to the hospital. Fortunately, B survives.

### Embodiment 17—Health and Wellness (SUBSYSTEM)

Nurse A also uses her anti-pathogenic wristbands as a body cooling system when she exercises.

### Embodiment 18—Medical Application (SUBSYSTEM)

Nurse A is also a 60-year-old menopausal woman who often uses her gym anti-pathogenic wristband to cool herself during hot flashes.

### Embodiment 19—Fire Ladder & Bucket Rescue (PRIMARY SYSTEM & SUBSYSTEM INTEGRATION)

Philadelphia fire truck ladder A is dispatched to a fire at 1776 Philadelphia Street because of a report of a building fire in an apartment complex. Truck A is advised that a ladder bucket will also be needed to rescue a ten-year-old little girl who is trapped in her apartment on the twelfth floor of the building. When ladder A arrives, B is crying for help from her apartment window.

All of the lower-level floors are fully engulfed with flames and thick black smoke that is now covering up a large section of the building and most of B who has been leaning out of the window and is choking and gasping for air. If B is not rescued within 5 minutes, she will likely succumb to the smoke and fire.

Fortunately, Philadelphia fire truck ladder A is the first truck on the east coast to be equipped with the new 360-degree Anti-Carcinogen Air-Force-Field Ladder Bucket System. The bucket opens up into a 6×6 platform and overhead mount that can hold two firemen and three people who may be insulated within a protectively enclosed 360-degee force field of air and/or water.

Two firemen are lifted up to the sixth floor. However, as they near the window to extract the girl B, large plumes of black smoke drift out of B's window and upwards from the apartment windows directly below B's apartment, totally engulfing the firemen, making it impossible to see anything. One of the firemen activates the system, which then creates a massive air bubble clearing around the ladder and the heroes are able to safely rescue the trapped little girl B.

### Embodiment 20—Evacuation of Contaminants From Structures (PRIMARY SYSTEM)

A class of kindergarten children are in the school district's first of its kind anti-pathogenic 30×20 classroom. There are several vents in the ceiling that are designed to extract germs up and away from the children in class. There are also several strategically angled vent openings on the floor and baseboards. Each vent is connected to a primary system export device.

Air that is pushed up and over the floor from the baseboard vents intercepts the air pushed up from the floor vents. Because the two air masses meet at a slight upward angle, a force field blanket of protective air forms above the floor of the classroom. The air is then forced upwards, pushing up dust, airborne pathogens, and contaminants. These contaminants are then sucked up into the vents of the ceiling.

Teacher A is writing the alphabet on the board in class when mother B knocks on the door. Teacher A opens the door and notices that mother B has brought her son C to school because C had missed the bus this morning. Teacher A thanks mother B for bringing her son C to class after which mother A leaves.

Teacher A tells B to take his usual seat at the table with the other children because they are learning their alphabets today. As B walks up to the table he suddenly stops and sneezes. However, before airborne pathogens from B's cold virus could reach the table to contaminate the other children, the vents on the floor and baseboards of the room diffuse and push the airborne germs up towards the ceiling where they are then sucked up into the filtered ceiling vents

### Embodiment 21—Anti-Pathogenic Air-Force-Field Insect Protection (SUBSYSTEM)

A is a police officer who works in Florida where there are a lot of mosquitos, gnats, and other insects. In the last few weeks, the number of mosquitos in Tallahassee has dramatically increased and several dozen community members have contracted the West Nile Virus and Malaria because of the recent rains and dirty settling water which is causing the mosquitos to lay more eggs.

Fortunately for A, his department recently issued him a pair of anti-pathogenic glasses due to the recent spike in Covid-19 and Tuberculosis cases in the county, which A and his squad mates have also been using on calls to blow away the pesky disease carrying insects from their faces and bodies.

### Embodiment 22—Anti-Pathogenic Attachment for a Fireman's Face Mask (SUBSYSTEM)

Fireman A is about to enter a burning, smoke-filled home. A checks the breathing apparatus attached to his face and eye mask shield system.

On each side of A's face mask are two (4 total) high powered industrial strength anti-pathogenic devices used to export air mass away from A's face at extremely high pressures. The air mass intake port systems of the devices are self-contained and sourced from an independent tank system on A's back.

A hears a whimpering dog inside the house and immediately runs into the home. A believes the sound is coming from the living room. However, A is partially blinded by the thick black smoke. A turns up his anti-pathogenic mask system to high power, causing a very powerful force field of air to form in front of and around A's face and head, allowing A to see. A is then able to locate the dog sitting in a cage in the living room. A rescues the dog and is able to safely return it to its owner.

### Embodiment 23—Anti-Smoking Gloves (SUBSYSTEM)

A is standing on the New York subway train where B has been openly smoking cigarettes. A sees several passengers putting on their anti-pathogenic glasses to keep from breathing in smoke from B's cigarette. An also notices most passengers have glasses that only have detachable import/export cylinders.

A does not have a pair of anti-pathogenic glasses. However, A is wearing a pair of anti-pathogenic gloves which she purchased for overseas travel, where smoking in business and restaurants are often permitted.

Each of A's gloves is equipped with a lightweight bracelet-like fitting that rotates around the circumference of A's wrist. Each wristband has an enclosure snap-in anchor design that receives a jet engine-shaped anti-pathogenic import/export device. The anchors are rotatable with the bracelet fittings around the top (i.e., over the back of the hand), sides, and bottom of the wrist.

A's gloves are also vented with a hollow hard material surface. The surface contours and follows the outside shape of A's palms and allows the rotatable devices to align with a hollow plastic opening under A's palm so that air mass which is imported and/or exported from the device is channeled under the palms and out through the palm vents. This allows A to open her hand(s) wide and expel air-mass from the palm area of her hands causing filtered air mass to push smoke and irritants away from her eyes and respiratory system.

A's anti-pathogenic glove device also has a system setting so that A can save her battery or charging system by disengaging or turning off the air filtration and/or ionization function of the device, and allowing the system to push smoke away while only using a fan function.

In the continuing embodiment, as smoke from B's cigarette travels across the cab towards A, she presses a button on the system which turns off the filter and ionizer. A then holds out both of her palms causing air mass from her gloves to fan away the smoke and push it back in the direction of B.

Embodiment relating to a cancer patient within a primary system home integrated with a bed, vehicle, bio-sensors, and wearable eyewear alert system.

A mother owns a house which is equipped as a primary system. The home is programmed to have command and control (via a central processing panel in the mother's bedroom) functionality over all subsystems within and extending beyond the primary system residence. The system may also employ a remote-control function by linking to a cell phone, tablet, laptop, etc., for user convenience.

The primary system home is responsible for activation, override, removal, integration, and default control over all subsystems and subsystem extensions in communication with it.

Within the primary system home is an electric vehicle which may or may not be in the garage, a pair of anti-pathogenic sunglasses, and an anti-pathogenic bed. The bed was prescribed by a physician and is covered under the family's medical insurance policy. This is because the woman's daughter is immunocompromised due to chemotherapy treatment as a result of a recent stage 2 cancer diagnosis.

Throughout the residence are several bio-sensors that detect specified levels of pathogens in the air. Bio-sensors are devices that detect and measure relevant clinical pathogens such as bacteria and viruses in the air and on surfaces.

The sensors in the home are linked to the primary system in the owner's bedroom. Therefore, all subsystem components are linked together for the purposes of program communication and instruction and are receptive to contaminant detection by any system component.

Attached to the outside of the residence, adjacent to the door frame (like a doorbell), is a 3×4 inch micron sensitive, anti-pathogenic filament intake receiver pad with a video monitoring alert system.

The receiver pad or port intake mechanism may be made of various materials and configurations known in the art. It may alternatively be attached inside the residence and may also be portable, similar to a handheld preliminary breath test device (PBT), administered by police officers to detect alcohol.

Additionally, the micron receiver may be housed within an audio-visual system. In another embodiment, the audio-visual system may be integrated within the anti-pathogenic receiver system employing a voice and/or an AI assisted sound recognition program in conjunction with pathogen testing by the receiver system for persons requesting ingress into the building structure.

In an embodiment, the receiver pad or other collection mechanism on or within the home or building structure, transmits viral and bacterial germs to a point of contact pathogen identifier for point of contact identification (to the degree possible).

At least one of a plurality of scientific identification means may be employed by the pathogen identifier. For example, specific DNA and/or RNA nucleonic molecular signature identifiers of the most common pathogens or contaminants may be preprogrammed into the pathogen identifier for immediate matching when the pathogen identifier unit receives the sample from the micron receiver.

In a first testing embodiment, the micron receiver or collection intake mechanism is in physical contact with the pathogen identifier and the results are communicated to the primary system or subsystem which recognizes the pathogenic signature and prompts the system to initiate an alert or communication sequence.

In a second testing embodiment, after the germ receiver or collection mechanism has stored the specimen, the pathogenic signature of the specimen is interpreted in real time and then transmitted to a medical testing facility for preliminary identification. The results are then communicated back to the residential primary system host for entry determination.

Additionally, an ionization charge or other pathogen neutralizing current or solution may come in physical or proximate contact with the contaminated receiver intake to promote safety by reducing the chances of cross contamination.

The detection results of several types of respiratory viruses and bacteria specimens are communicated to the primary system and may also be communicated to all subsystem components.

A visitor to the house desiring entry may be prompted to expel air onto or into a receiver by coughing or blowing on it with sufficient force to collect a specimen for rapid testing and preliminary identification.

A negative or positive test may determine entry into at least one residential primary system and/or subsystem component by requiring a visitor to be cleared prior to entry into the primary system home or other building structure.

Once the specimen is analyzed and a preliminary positive or negative result is determined, the primary system home, building, tent etc., may communicate the result(s) to any other integrated component of the system for entry determination.

Any entry into the home after a specimen is received, processed, and preliminarily identified as negative or positive, may be at the discretion of the resident or caretaker of the home or building structure.

Alternatively, entry may be determined via automation, remote video access (e.g., manual video or photo identification), facial recognition, or other types of machine learning, AI, or other computer implementation.

In an embodiment, simply requiring a person to put on a mask after a positive test may be sufficient to permit entry into a protected structure.

In another embodiment, immediately after a positive test is detected, an audio or video feed from a camera placed outside the structure enclosure door, appears on the cell phone of the homeowner or building custodian, preferentially in real time.

Access may also be approved from a remote or distal location or with a video, or in conjunction with a home video or audio-video combination monitoring system. A positive test can alert any of the system components of the result.

For example, a wearable anti-pathogenic wristwatch system, while in a different state or country, may receive a door test result notification via a live video stream feed or a recording from a camera in proximity of the door.

This would afford an opportunity for a person who had preliminarily tested positive at the door or access point to seek medical confirmation or treatment, prior to accessing any protected residential, medical, or commercial structure while simultaneously protecting persons inside from infection and contamination.

In an example use of this embodiment, two church members knock on a residential door asking a homeowner inside if they can come inside a residence and talk about religion. The homeowner agrees. However, the homeowner asks both individuals to first blow onto the exposed receiver adjacent to the door. Both individuals comply and blow onto the exposed receiver pad. Thirty seconds later, the homeowner on the other side of the door receives a cell phone text message indicating possible Covid-19 infection. The two church members are then denied entry.

The system is beneficial because it not only protects people inside a residential or commercial structure against pathogenic exposure but also warns others who might be infected to use a protective barrier such as a mask or face shield, and to seek the necessary medical treatment to prevent the spreading of air-borne pathogens.

The system can also be integrated with a vehicle. To continue the example, the mother places on a pair of medically prescribed anti-pathogenic glasses which came as an accessory with her new Tesla. She enters the garage and gets into the family vehicle which is programmed as a secondary primary system. She then drives to the food store. While inside the store, her anti-pathogenic glasses vibrate. Her cell phone, which is linked to her anti-pathogenic glasses by a mobile application, begins to ring before displaying the following message:
“POSSIBLE EXPOSURE FROM A HOME VISITOR. CONTACT YOUR DAUGHTER ASAP.” The mother calls her sick daughter at home but there is no answer. Once inside the Tesla, an audible message announces:
“POSSIBLE EXPOSURE AT HOME FRONT DOOR ENTRY.”

Additionally, the vehicle dashboard displays:
“4:37 pm: Covid-19 exposure detected at front door entry.”

The mother checks the audio video feed from her cell phone which shows a male wearing a white t-shirt, black sweats, and a baseball hat standing at her front door knocking and requesting entry into her home.

She can also hear (via the home audio system through her cell phone), as her sick daughter asks the man, who the mother recognizes as a friend they are expecting for dinner, to blow onto the pathogen receiver adjacent to the door.

The guest blows on the receiver. After a minute, the mother hears her daughter ask the man to place on a mask because the system had preliminarily detected he has or has recently had “a cold virus.” The guest puts on a mask. The daughter is then seen on her mother's cell phone feed, opening the door and letting the guest inside the house.

The mother drives home and parks her vehicle in the garage. She walks inside the house and notices the guest and her immunocompromised daughter are wearing masks. The mother walks upstairs and into her daughter's bedroom. She notices all of the anti-pathogenic cylinders attached to her daughter's medically prescribed bed are operating at full thrust capacity; pushing out a 360-degree curtain of air away from all sides of the bed enclosure, including from under the bed.

The bed subsystem had activated because within fifteen seconds of the positive door test indication by the family friend, the front door motion entry sensor was triggered. This indicated possible entry into the residence by an infected person in proximity of her immunocompromised daughter. She also noticed that the negative ion system integrated with the bed had also been activated.

It should be understood that negative ionic charges occur in nature and have been scientifically documented and proven to attach to, weigh down (causing pathogens to fall to the ground) and kill or neutralize the air-borne pathogens.

In addition to the capability of the system to alert and make notifications, cell phones, tablets, and/or computer systems may be optionally integrated within the system for the purpose of remote system activation, system disengagement, communications, audio video notification, live feed communications, etc.

Unlike audio visual remote access doorbell entry notification and identification systems currently practiced in the art (e.g., Amazon's RING, Google's NEST HELLO, Arlo ESSENTIAL, etc.), employing portable or remote cell phones, computers, tablets etc.: the system currently being introduced relates to an anti-pathogenic protective primary system enclosure, the enclosure being designed to protect a specified area such as a residential home, residential structure, commercial building, government building, scientific building, or medical building.

In regard to this system, unlike the aforementioned doorbell audio video platforms, the current system's requires visitors to expel air on a receiver mechanism for contaminate pathogen identification prior to a primary system entry.

### Primary System Vehicle With Anti-Pathogenic Monitoring Capability

A primary system vehicle or enclosure may be programmed to control at least one optionally integrated, wearable, or portably transportable subsystem device and/or at least one secondary primary system (programmed to be subordinate to the parent system) which captures, reads, interprets, translates, or communicates the presence or probability of airborne pathogens and/or contaminants proximate to or within at least one specified enclosure or area designed to protect humans, animals, or other living organisms subject to anti-pathogenic deterrence.

A primary system autonomous vehicle or enclosure, may also be removed as the unifying primary system while the system continues to operate via at least one secondary primary system activation or priority override default program during incidences such as: catastrophic and minor primary system failures, maintenance, system redeployment or reconfiguration, etc.

In an embodiment, during flu season, the mother of an infant is parked in a store parking lot inside a primary system autonomous vehicle. The vehicle is in digital communication with a secondary (non-wearable) primary system, and anti-pathogenic infant vehicle seat carrier.

The vehicle seat has an infant inside and is being carried by a babysitter who is also the family nanny and is inside a shopping mall 25 miles away from the primary system vehicle. The babysitter is wearing an anti-pathogenic infant arm guard which has been programmed to receive a vibration alert, followed by a digital display message on the LCD of the armguard, which may originate from the vehicle.

The babysitter walks into a music store carrying the infant vehicle seat carrier in her left hand. On her right forearm, she wears the anti-pathogenic infant armguard given to her by the infant's parents before taking the infant.

The subsystem (or secondary primary subsystem if no primary system default occurred), baby carrier is equipped with an anti-pathogenic vent tubing system. The tubing is internally integrated within the semi-circumferential rim opening where the infant is placed in and retrieved from the hard-shell of the subsystem carrier enclosure.

There are several ways in which anti-pathogenic import/export mechanisms may be deployed within an infant and baby carrier system enclosure. However, in this particular embodiment, the infant vehicle seat carrier is deployed in the following manner:
1). Air is imported from the rear (in back of the carrier) shell of the carrier enclosure towards the front shell opening though several inlets that are physically integrated within the system frame.
2). The air is then filtered and optionally ionized and/or treated within the processing cylinder of the anti-pathogenic system.
3). After importation, ionization, and/or treatment, the air is charged and/or compressed via the application of an internal fan or impeller mechanism.
4). The air is then directly exported (or adjustably sprayed) from at least one to a plurality of semi-circumferential vents around the enclosure opening. This results in an “Air force-field” of protection in front of the infant enclosure.

The babysitter is wearing an anti-pathogenic infant armguard which is optionally equipped with a bio-sensor or bio-chip mechanism which can detect pathogens, such as influenza, covid, and cold viruses.

Communication from the bio-sensor or bio-chip mechanism to the armguard would, if so programmed, cause the armguard to audibly emit at least one tonal warning or frequency upon the detection of specified levels of airborne pathogens, carcinogens, or other contaminants in proximity to the infant carrier. The activation and alert from the armguard or any subsystem component would also notify the primary system vehicle 25 miles away.

Any type of warning in the art may suffice, including: a verbal warning, a vibration warning, or any combinations of warning and/or vibration sequences.

System detection warnings may also be relayed via any unified component of the primary system, including devices with limited primary or subsystem programmability, such as cell phones, tablets, computers, audio-visual systems, etc.

However, in the continuing embodiment relating to the infant carrier, at the time the armguard was purchased, the bio sensor model had not been available. Therefore, the parent of the infant, instead purchased a model equipped with an accelerometer (motion detection circuit or chip). Without warning, a man 20 feet away begins to repeatedly sneeze in the direction of the babysitter and infant.

The sitter reacts to the pathogenic attack by shaking the infant armguard 3 repetitive times, causing the armguard to activate by exporting air.

The babysitter then starts making continuous circular motions in front of her face and chest using the exported air from the armguard to deflect airborne pathogens away from her respiratory system and clothing.

The activation of the armguard sends a digital signal to the primary system vehicle 25 miles away, and all integrated systems as follows:

The primary system vehicle receives an indication of the motion detection pattern output by the babysitter and calculates the distance the sitter is away from the infant carrier, e.g., using GPS or another positioning locating system practiced in the art.

The primary system vehicle determines the infant carrier was approximately two feet away from the babysitter at the time the man sneezed.

In an alternative embodiment, machine learning techniques relating to voice or sound recognition may also be used. For example, the infant armguard or baby carrier system may recognize the sound of a person sneezing or coughing, prompting system activation.

The rapid motions of the armguard may simultaneously trigger the initiation of the infant carrier's anti-pathogenic system. Additionally, the mother inside the vehicle may also trigger a fail-safe override by the primary system vehicle to ensure activation of the carrier system or other subsystem component.

The carrier protecting the infant activates and deflects thousands of microscopic Covid-19 and Influenza pathogens away from the infant within the vehicle seat carrier.

The carrier sends a digital signal to the LCD screen on the babysitter's armguard which reads:
“10:10 AM: CYLINDER ACTIVATION. POSSIBLE INFANT EXPOSURE. CONSIDER PREVENTIVE CARE.”

In the same continuing embodiment, the LCD screen on the dashboard of the primary system vehicle that the mother was driving simultaneously reads:
“10:10 AM: CYLINDER ACTIVATION. POSSIBLE INFANT EXPOSURE. CONSIDER PREVENTIVE CARE.”

It should be understood that the system can be programmed so that any system-wide notification sequence can be initiated by any primary system or subsystem component, that is integrated with the system to communicate with other system components for the purpose of contaminant notification.

### Anti-Pathogenic Bed and Vehicle Systems

In an embodiment, a primary (parent) system infant bassinet may be programmed to control at least one optionally integrated, wearable, or portably transportable subsystem device and/or at least one secondary primary system (programmed to be subordinate to the parent system) which detects and communicates the presence of or probability of airborne pathogens and/or contaminants proximate to or within at least one specified enclosure designed to protect humans, animals, or living organisms subject to anti-pathogenic system deterrence.

In a similar embodiment, a primary (parent) system bed may be programmed to control at least one optionally integrated, wearable, or portably transportable subsystem device and/or at least one secondary primary system (programmed to be subordinate to the parent system) which detects and communicates the presence of or probability of airborne pathogens and/or contaminants proximate to or within at least one specified enclosure designed to protect humans, animals, or living organisms subject to anti-pathogenic system deterrence.

In another embodiment, a primary (parent) system vehicle may be programmed to control at least one optionally integrated, wearable, or portably transportable subsystem device and/or at least one secondary primary system (programmed to be subordinate to the parent system) which detects and communicates the presence of or probability of airborne pathogens and/or contaminants proximate to or within at least one specified enclosure designed to protect humans, animals, or living organisms subject to anti-pathogenic system deterrence.

A primary system bassinet, bed, or vehicle enclosure comprises at least one stationary or adjustably walled partition or enclosure attachment. In the preferred embodiments, the partition or enclosure attachment of the primary system(s) are integrated with the body support portion of the enclosure.

The import/export mechanism(s) (thrust cylinders, pylons, etc.) may be conveniently detachable from the primary system and deployed around, beneath, or above a protected person within a primary system bed, bassinet, vehicle, or remote subsystem enclosure integration that is in communication with the primary system enclosure.

Additionally, wearable subsystems, through various SAAS or programmed priority placement override communicating programs may also (preferably temporarily) act as secondary primary systems that are programmed to initiate a warning signal, phone relay communication, radio signal, digital signal, social media notification, AI facilitated notification, or other types of notifications or warnings in the art to at least one individual interacting with the system.

It should be of note that non-wearable anti-pathogenic pylons or system devices may also act as secondary primary systems or as interchangeable subsystem components.

This is because each subsystem component not permanently integrated into the bed enclosure may be detachable and deployed independently at strategic locations around the patient or protected person's bed or enclosure.

These strategic protection points may also be placed at medically designated or prescribed distances to prevent airborne pathogens from infecting protected individuals.

Additionally, anti-pathogenic system devices, cylinders, pylons, etc., may be manually or remotely focus adjusted at or near the base of the export vent.

The system may also be adjusted or programmed to export air mass at multiple directional angles away from a bed, bassinet, crib, vehicle seat system, or other protected enclosure.

Additionally, mass may be expelled vertically, horizontally, laterally, or in a cross-sectional direction and/or around, about, below, and/or above (up to 360 circular degrees), away from a primary or secondary system enclosure.

This would permit air mass to spread out and push pathogens and contaminants over a wide area away from a protected person within the enclosure.

For example, one benefit of upward exportation of air mass (if system devices are adjustably focused to do so), is that when air mass is simultaneously pushed upward from vents, cylinders, tubes, pylons, etc., on all sides of a person within the protected enclosure, (for example, an upward funnel air may push up from all sides of a medical bed, bassinet, incubator, etc.) the crest, or expanded skirt area of the funnel peaks above the enclosure, where all protected points meet, forming a unified cloud of mass, similar to a dissipating cloud shield above the enclosure.

Any number of anti-pathogenic cylinders may line the outside of an enclosure and may also be detachable from the enclosure. Additionally, the system devices may be of various shapes and sizes and are optionally vented (see FIGS. 2-3) to the outside or within the enclosure.

In an embodiment, the devices may be rotatable up to 360 degrees to facilitate the exportation of mass in all directions away from or across an opening or doorway within, above, below or flush with the enclosure entry or exiting space (i.e., a home baby crib enclosure, a home bassinet enclosure or proximate to an infant vehicle seat carrier system).

In a specific embodiment, within a hospice care facility, eight 1×2 foot anti-pathogenic cylinder or pylon configured system devices are attached on the outside left (4) and outside right (4) partition walls. There are also four devices on the outside head (2) and outside foot (2) partition of the bed. The four cylinders that are attached on the right-side partition export air outwardly in a wide cone-shaped funnel trajectory, permitting midrange, lower range, and upper range protection from intrusive germs which may descend towards the bed from various angles.

The export cylinder vents on the opposite side partition wall, as well as the ones at the head and foot are configured in a similar manner. However, in this specified embodiment, the primary system bed is not equipped with anti-pathogenic system devices underneath the bed system.

Due to this configuration, when the bed system is activated, an immunocompromised patient is protected within a shield, or ‘Air force-field,’ of anti-pathogenic protection.

In an embodiment, four 2×2 individual pylons are placed in a square at four points around a newborn infant in a bassinet within a hospital maternity ward. Collectively, the four anti-pathogenic pylons placed around the bassinet form a single (or parent) primary system enclosure.

Although the newborn is not premature, she was born with a possible lung defect, so she is placed in an isolation room in the middle of the anti-pathogenic pylons. Only recently tested medical staff are permitted within 6 feet of the newborn for the first week after birth. The medical staff members are each required to blow upon a pathogen receiver or intake mechanism (essentially a type of breath doorbell) which detects if the staff member has been infected before gaining access to the immunocompromised newborn.

As previously cited and exemplified within the disclosure of this application, embodiments are exhibited whereby a medical or standard bed enclosure primary system may also be programmed as a subsystem component (not a wearable extension) of a larger primary system (e.g., house, building, vehicle, etc.)

It has also been exemplified by the introduction of several previous embodiments, that larger primary system enclosures may employ a parent or centralized, command-and-control health and wellness instruction and/or patient monitoring system capacity.

For example, in an example embodiment, a primary system home would be the controlling system authority over a vehicle, a baby crib, an infant armguard, and a pair of anti-pathogenic glasses.

However, the vehicle, while travelling on a family road trip (transporting an infant in a car seat, a teenager wearing a fashion model anti-pathogenic armguard and a mother wearing a pair of anti-pathogenic glasses) would take over as the primary anti-pathogenic system (unless otherwise programmed). The other components would be subsystems.

For the purpose of understanding, beds and bed enclosures may be found in homes, residential buildings, treatment center buildings, condominium buildings, hotel buildings, office buildings, medical buildings, therapeutic and spa buildings as well as in scientific research and experimental buildings. Beds and bed enclosures may also be found in temporary abodes and dwellings such as makeshift medical tents and shelters.

Beds and bed enclosures may be deployed within a road driven vehicle (e.g., automobile, camper, trailer) or within an air transportable vehicle such as a helicopter, or airplane. Beds and enclosures may also be manually driven, pushed, pulled, and/or electronically driven. Additionally, beds and bed enclosures may also be remotely driven or relocated via the application of a machine learning (AI) learning program or other automation system.

Medical beds and bed enclosures may also be transportable or incorporated within a vehicle and include: infant incubation beds, infant bassinet beds, patient treatment beds, bariatric beds, as well as patient operation and medical procedure and evaluation platforms.

An anti-pathogenic medical treatment bed enclosure primary system may also be deployed within or transported by an emergency services vehicle such as an ambulance, public safety vehicle or within a standard vehicle. The system may also integrate with a primary system medical airlift or standard air transport vehicle such as a helicopter or airplane.

### AI System Integration With Anti-Pathogenic Vehicle and Infant Monitoring

In an embodiment A is the driver of an autonomous vehicle that is traveling on a freeway. B who is a passenger tells A she needs to use the rest room. A stops vehicle C (C is a primary system vehicle) at a rest area and recharges vehicle C while B, who is wearing a pair of anti-pathogenic glasses D (D's glasses are subsystems of vehicle C) with a bio-chip sensor and SAAS enhancement program, exits vehicle C and walks into the store.

In the continuing embodiment, babysitter E is at home watching A and B's infant who is asleep in transparently walled bassinet F (F is the second primary system integration and enclosure). Bassinet F is also in communication with subsystem glasses D (and optionally with primary system vehicle C).

The enclosure section and shape of bassinet F is transparent and rectangularly walled, similar to a hospital bassinet, with a base, two side walls and two shorter head and foot walls. The top section of bassinet F is uncovered for the placement and removal of infants into and out of the enclosure.

Four separately removable, 3-inch-wide rectangular anti-pathogenic thrust cylinder tubes are vented upward (the top export side faces the ceiling). The opposite side faces downward (the bottom import side faces the floor).

When bassinet F receives an activation signal air is imported into the cylinder tubes, filtered, and may be ionized and/or treated before being exported and sprayed upward along at least one side of enclosure bassinet F.

The exported air is initially narrowly coned but may also be adjustably focused and controlled at the base. In the preferred embodiment, when the system is activated, air from the cylinders of bassinette F will rise upwards towards the top portion of the open enclosure and spray into a wide funnel above the opening of bassinet F.

The velocity of the air is such that as it is forced upwards it becomes less concentrated and forms a resistant anti-contaminant funnel or skirted barrier above the infant. This air is able to push contaminants away which may have otherwise infected the infant.

In the continuing embodiment, several moments after entering the store B's anti-pathogenic glasses D (equipped with a bio sensor) detect coronavirus, influenza, tuberculosis, and carcinogens in the air. B's cell phone (linked to B's glasses) vibrates to warn B she may be exposed. B's glasses D emit a tone which also warns B she should either exit the establishment or put on a mask.

At the same time A who is sitting in vehicle C receives an audible message from vehicle C that B may have been exposed to airborne pathogens.

The dashboard on vehicle C displays the following: B POSSIBLY EXPOSED FOR 5 MINUTES TO: 1. Covid-19 2. Influenza 3. Tuberculosis 4. Smoke.

B exits and tries to gain entry into the passenger door but discovers it has automatically locked because primary system vehicle C detected from glasses D that B has been exposed to pathogens. However, A notices B had placed on a mask and unlocks the door, allowing B to enter the vehicle.

Babysitter E is at home watching the infant sleep in bassinet F when she receives an text message on her cell phone from B's cell phone that she (B) had been possibly exposed to pathogens and to keep the baby away from her when she and A arrive home. Thirty minutes later A and B walk into the house.

Once inside the residence a proximity signal from B's anti-pathogenic glasses D transmits a signal to a receiver in bassinette F causing the anti-pathogenic devices in the bassinet to turn on to protect the infant.

### Additional Embodiments

In another embodiment a man with a wearable subsystem wrist-watch leaves his autonomous vehicle and enterers into a bar. While inside the bar, an AI (machine learning) program in his watch recognizes that out of the 50 people inside the restaurant, 20 have all sneezed approximately two times each within the first 5 minutes.

In another embodiment a truck driver wearing pair of prescription anti-pathogenic eye glasses equipped with a bio sensor receives a signal on his cell phone while he is inside a store for a delivery. The phone then displays a SMS message which indicates his partner, who is waiting for him inside the truck, has sneezed five times in the span of 5 minutes.

After the driver is alerted, he walks to the truck and when he tries to open the door, he noticed the lock delays him from opening the door for 10 seconds. He then places on a mask and enters the truck.

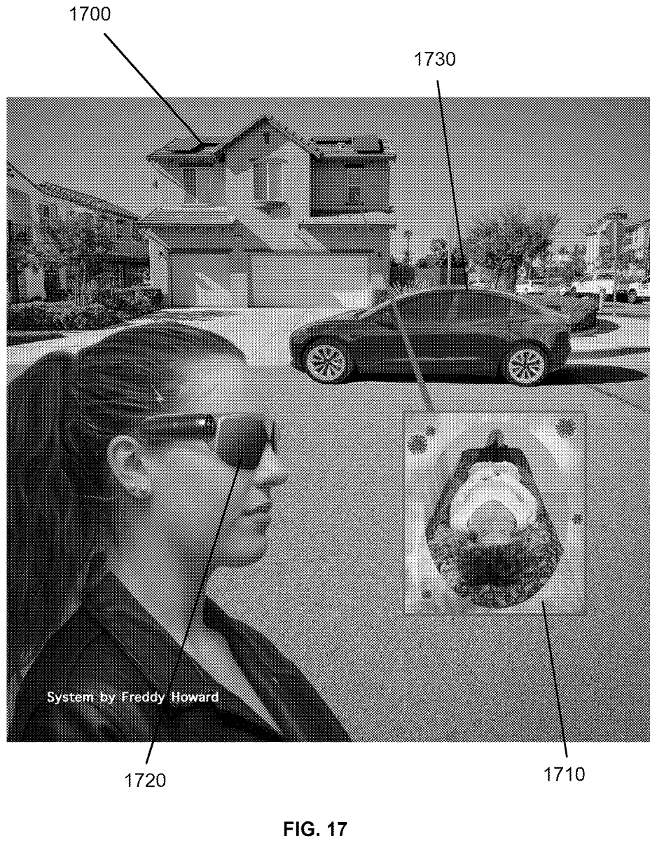

FIGS. 17-26 illustrate an example of a primary system anti-pathogenic residential home 1700 with several integrated subsystems. Some of the subsystems may have bio-sensors while others may not.

The anti-pathogenic subsystems inside and/or outside the home may include: a remote anti-pathogenic audio-visual door entry alert system, an anti-pathogenic breath test door entry request device (a sort of breath doorbell), an anti-pathogenic bed 1710 for protecting an immunocompromised patient living in the home, a pair of anti-pathogenic eyeglasses 1720, and an anti-pathogenic vehicle 1730 with a dashboard contaminant identifier display and audio-visual pathogen deny-permit entry system.

The vehicle 1730 is programmed to be in communication with the house primary system. The vehicle 1730 may also serve as the secondary primary system if there is an issue with the primary system.

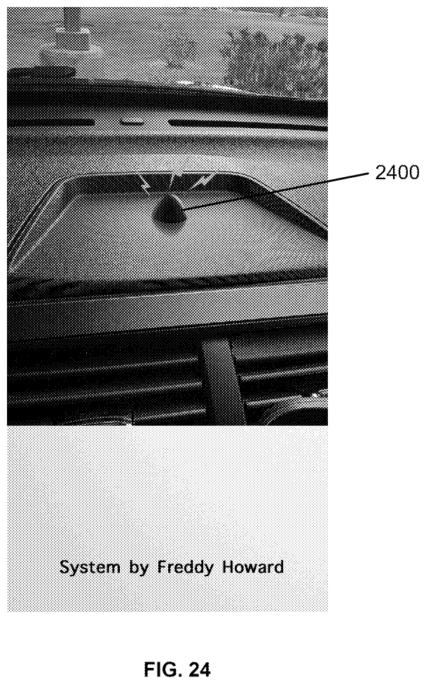

The vehicle system also includes a bio-sensor (see FIG. 24) integrated with a digital receiver/transmitter located on the top middle portion of the vehicle dashboard.

The anti-pathogenic glasses 1720 are equipped with a bio-sensor that can detect pathogens and other contaminants.

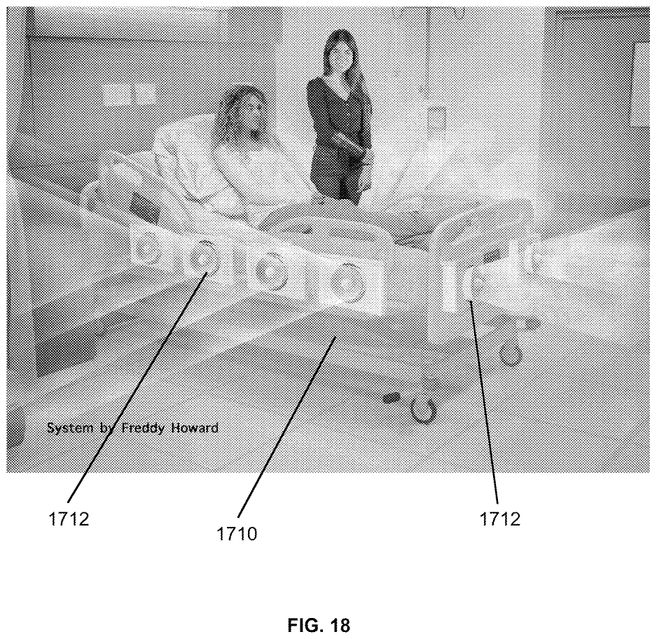

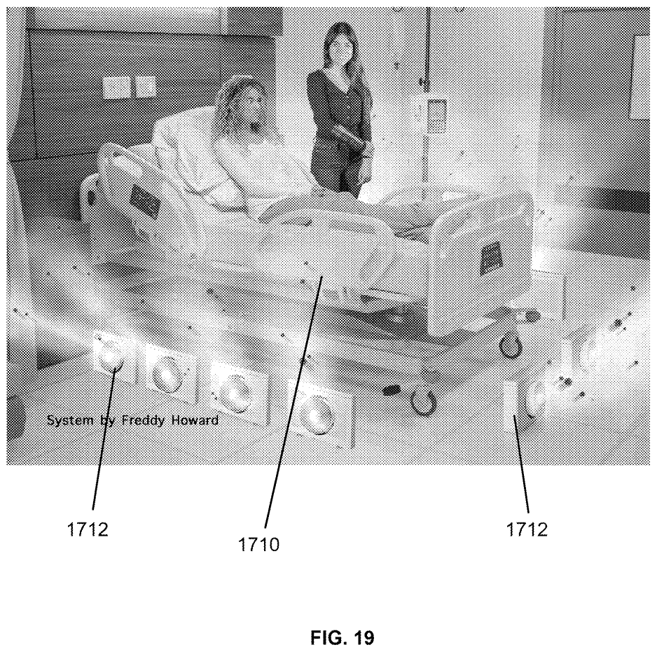

FIG. 18 shows the anti-pathogenic bed 1710 inside the residence 1700 of FIG. 17. This particular system does not have a bio-sensor, but may automatically start if any of the linked subsystems (e.g., vehicle 1730, glasses 1720, breath doorbell, etc.) detect pathogens.

The bed 1710 was prescribed by a doctor for a young woman diagnosed with cancer. The bed is not limited to medical purposes, e. g, the bed may be a typical home bed, cot, or recliner.

The mass export vents 1712 integrated with the bed may also be detached from the bed and placed on the floor around the bed at various locations to push pathogens away from the bed, as is shown in FIG. 19.

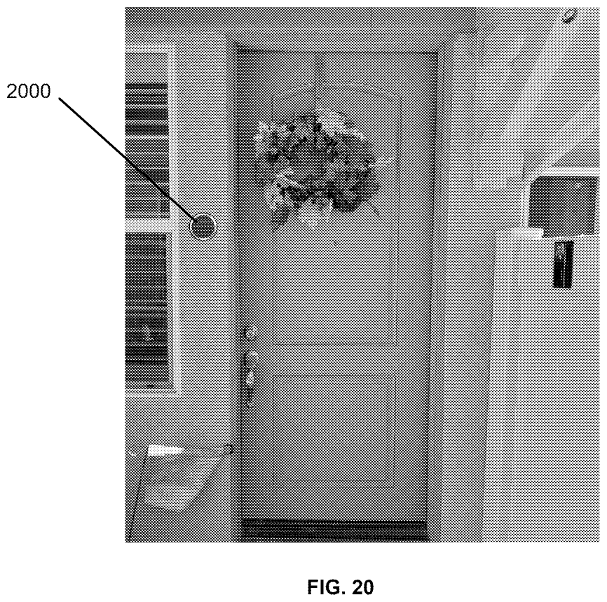

FIG. 20 illustrates an anti-pathogenic filament intake receiver pad 2000 (essentially a breath doorbell) which a person may be required to blow or cough on before being permitted into the house or other building structure. This type of doorbell system may also be used at medical facilities.

The pad 2000 may be various shapes, sizes, and materials, and is preferably located on the outside part of the house near the door, similar to a doorbell but may also be used on fences and walls around the curtilage of a house or building.

In an embodiment, the receiver pad 2000 has an ionizer intermittently discharging ions to kill or neutralize airborne pathogens collected on and within it for safe disposal of internal filters and to prevent cross contamination of persons touching it.

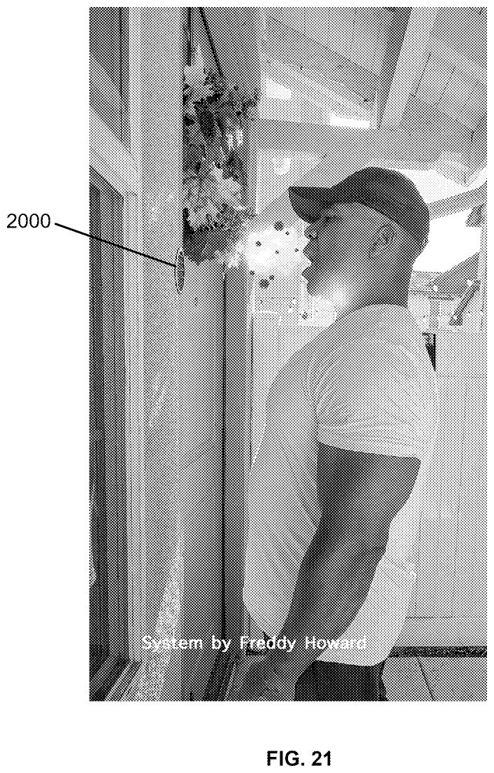

FIG. 21 illustrates a man who has requested entry into the anti-pathogenic protected house where the immunocompromised daughter of the homeowner lives. The sick daughter asks the man to first blow into onto the pathogen doorbell and the man takes a deep breath before blowing hard onto the pathogen receiver 2000.

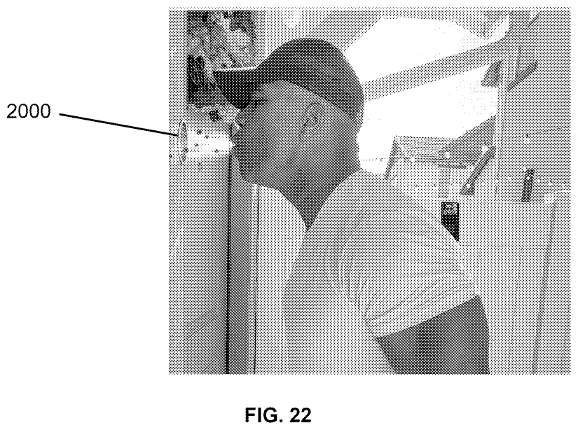

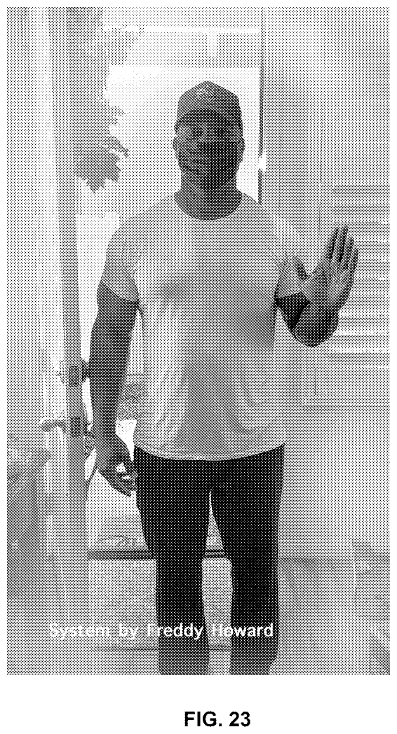

FIG. 22 demonstrates the man blowing onto or into the receiver pad 2000 where the man's breath is processed for a preliminary or confirmation test result. The test can be local or sent to a testing facility. If the test is negative, the man may be permitted into the home, as shown in FIG. 23.

FIG. 24 illustrates a bio-sensor 2400 which detects pathogens and other contaminants within or proximate to the vehicle 1730. The bio-sensor may be integrated within a dashboard transmitter housing and is in communication with the glasses 1720 and other connected subsystems for pathogen alert and identification.

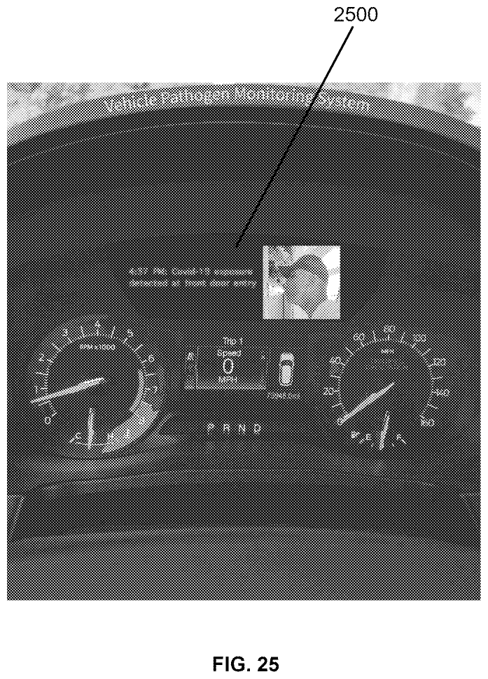

FIG. 25 illustrates the digital dashboard display 2500 of the vehicle 1730 which has activated in response to the detection of pathogens by the pad 2000. However, activation may also occur in response to the detection of pathogens by the eyeglasses 1720 worn by the homeowner while outside of the vehicle, or by other connected subsystems such as the bed 1710. The display on FIG. 25 reads—“4:37 PM: Covid-19 exposure detected at front door entry” and shows an image or video of the person using the pad.

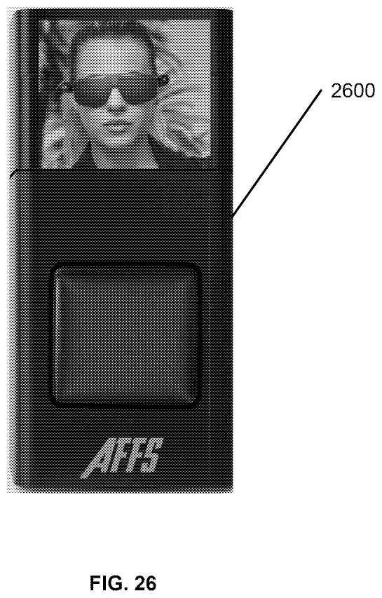

FIG. 26 illustrates an audio-video monitoring system 2600 which is activated after receiving a signal from the receiver pad 2000. The signal may indicate the presence of a person at the door and/or the results of any breath test.

A device (e.g., a cell phone) carried by the homeowner is configured to communicate with and display messages received from the various subsystems. For example, the device receives messages in response to the activation of the receiver pad 2000 and any breath test results.

Activation and subsequent notification of the anti-pathogenic systems in the house may occur as a result of motion detectors (not shown) proximate to a door, showing entry into a residence quickly or at a specific time after a positive door breath test is received.

### Embodiment 24—Anti-Pathogenic Gloves Smart-Home & Building Sanitizing

A is the owner of a level 3 primary system anti-pathogenic equipped residential smart-home. A's level 3 system is only integrated with his electric vehicle and does not communicate with other system extensions. However, level 1 and 2 configurations may communicate with other anti-pathogenic primary and subsystem devices and system extensions.

In an alternative embodiment, A's level 1 and/or level 2 connected anti-pathogenic smart-home and smart-building notification platform may communicate with other primary system structures including A's electric vehicle and his wife (B)'s new electric vehicle.

A's system may also communicate with wearable or portable subsystem component extension devices that employ level 1, 2 or 3 monitoring and notification capabilities (e.g., cell phones, computers, audio-visual devices, anti-pathogenic armguards, glasses, watches, gloves, baby carriers, infant bassinets, bed systems etc.), if programmed or configured to receive contaminant notifications from A's primary system residential home or vehicle. This may also occur within anti-pathogenic equipped smart-buildings such as commercial, medical or government building structures.

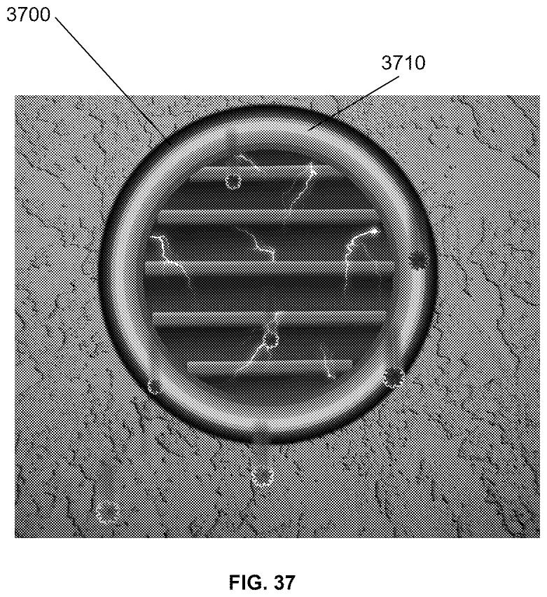

Visitors at A's front door are prompted to blow respiratory air onto the bio receiver 3700, illustrated in FIG. 37 (other models may require fingerprint scans, AI identification and/or AI predicative pattern exposure calculations, voice and sound recognition programs etc.).

A's family members and anyone inside A's home or vehicle (if so programmed) receive a contaminant notification message (audio-visual, audio or visual on A's vehicle display or on A's home television monitoring system) from the bio sensor relay integrated with A's “Breath Doorbell” system.

The system can alert A and members of A's family if the person requesting entry into their home has preliminarily tested positive or may have a probability of testing (or confirming or dispelling) positive or negative after their bio sample has been submitted and analyzed.

A's particular breath doorbell utilizes a point of contact testing method where germs are immediately analyzed upon contacting the surface of the bio sensor receiver filament.

However, in an alternate testing embodiment, results by A's breath doorbell device are communicated in real-time via remote off-site testing by medical diagnostic or scientific research centers in communication with A's contaminate monitoring system.

In a similar embodiment A's breath doorbell displays results utilizing immediate downloadable identification sample comparisons of the most common pathogen strains reported (i.e., influenza, the common cold etc.) by an agency such as the U.S. Center for Disease Control (CDC) or foreign equivalent for a predictive contagious season, year, or other period of time.

Specified models may also employ an air import suction or vacuum method where the device's fan or impeller mechanism sucks in (or imports) pathogens, contaminants, and/or biological samples containing genetic material to be analyzed, tested, and neutralized.

A had a Christmas party at his home yesterday and about 40 people attended. All attendees were required to voluntarily expel respiratory air onto the bio receiver filament before being permitted inside because it was flu season.

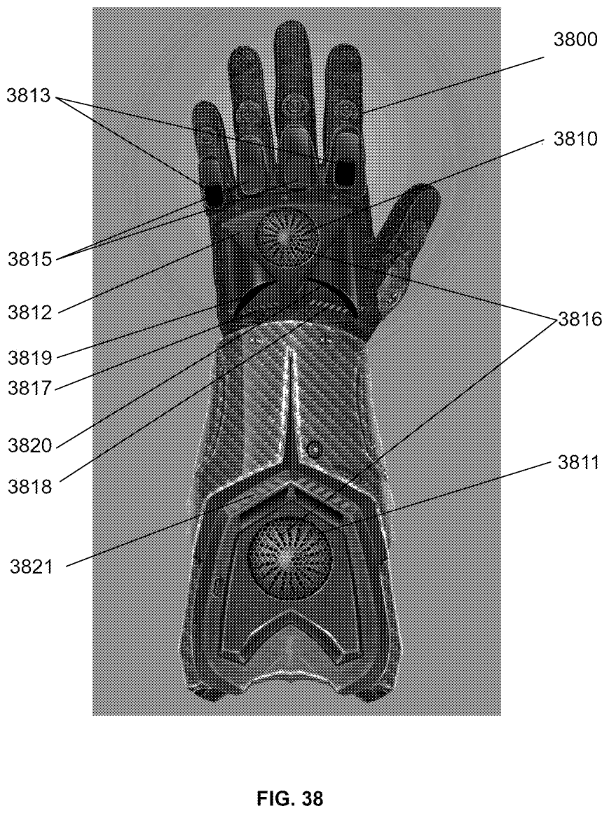

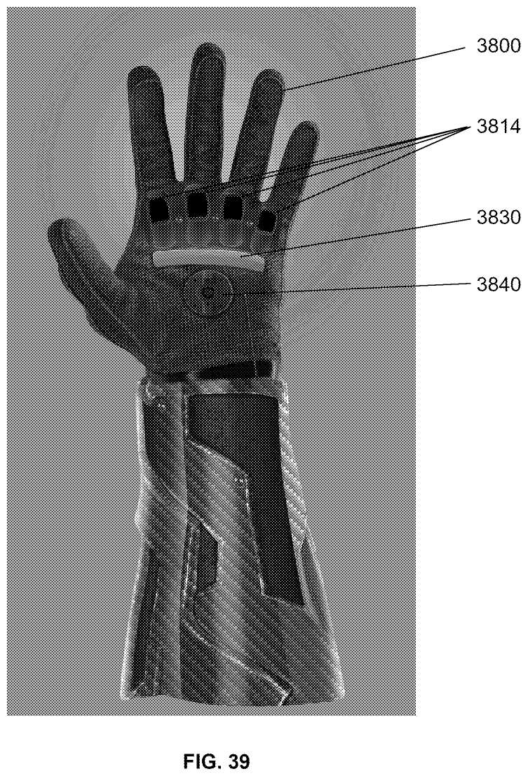

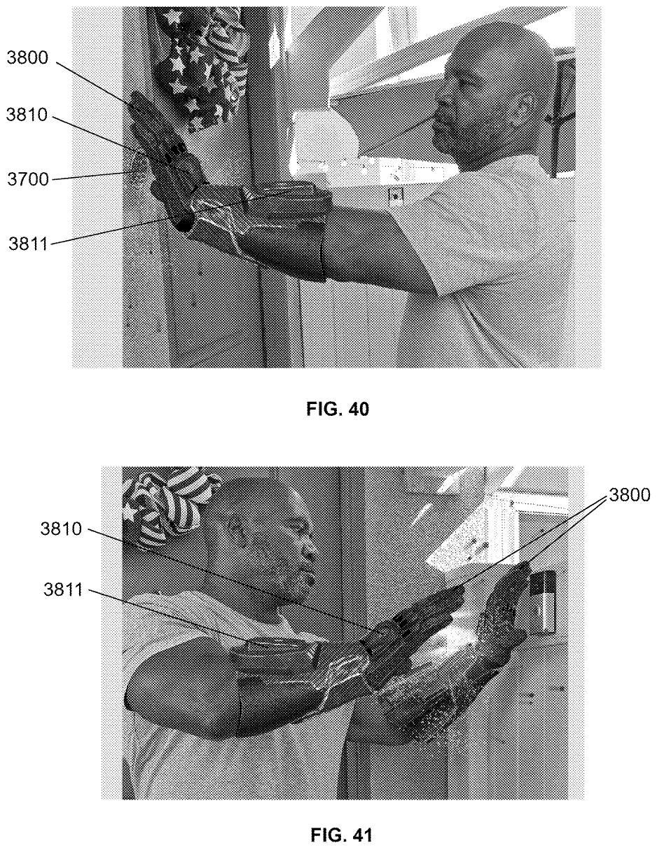

After the party, A decided to sanitize his system as recommended. A utilized his pair of industrial strength, heavy-duty, carbon-fiber, anti-pathogenic hard-shell gloves. These gloves 3800 are illustrated in FIGS. 38 and 39, with use cases illustrated in FIGS. 40 and 41. These gloves came as part of a smart-home vehicle integration package when he purchased his new electric vehicle.

In an embodiment, the breath doorbell can be configured to sanitize surfaces proximate to the receiver, to reduce the risk of cross-contamination.

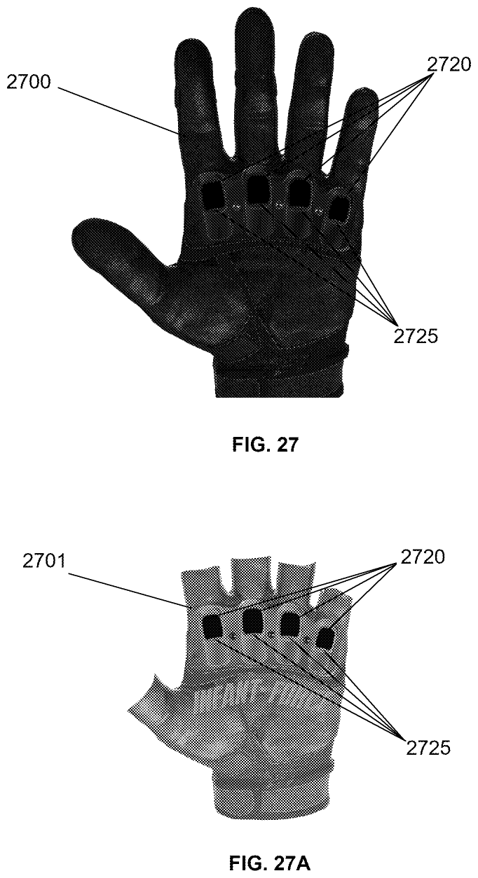

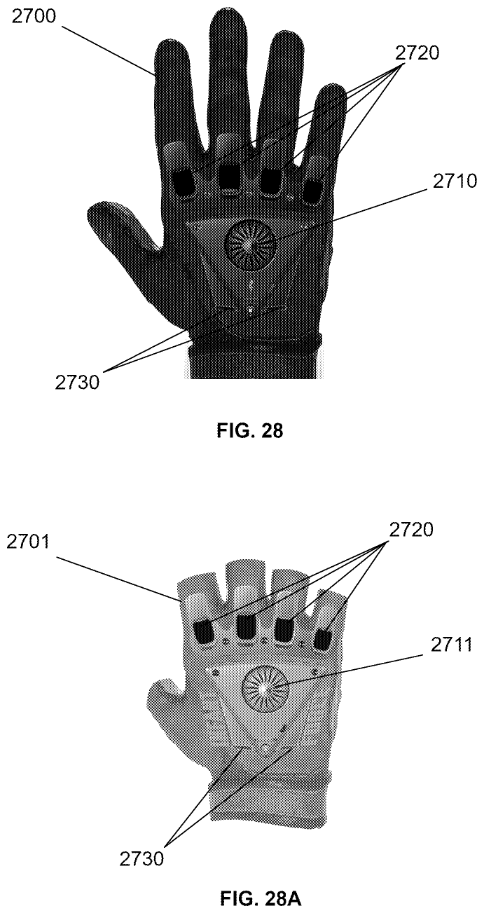

A's gloves are full fingered and significantly larger than the light-weight fingerless INFANT-FORCE gloves (illustrated in FIGS. 27A and 28A) and the black full-fingered antipathogenic gloves (illustrated in FIGS. 27 and 28) that A's wife B had purchased separately. His wife B had purchased them because she expects her one-month-old infant (E) to be around family and friends who may be sick or have been exposed to germs.

B was aware that the lightweight fingerless anti-pathogenic gloves were made for mothers who want to protect their babies but who also want to touch and feel them as they are nursing, sanitizing bottles, etc. They may also be used to push away smoke, e.g., during wild-fire season and for when people who smoke (like B's mother) or carry odors come to visit.

A walked over to his gloves which were on a counter charging from a wall socket to the USB port in the motor casing assembly on the hand portion of each glove. A's particular gloves are heavy-duty industrial strength models. A's gloves are C-F-M (Commercial Food & Medical) rated and certified for sanitizing and disinfecting contaminated commercial scale kitchens as well as medical facility waiting rooms, devices and appliances.

Unlike B's gloves which have only one fan (2710, 2711) on the back hand portion of each glove, A's industrial strength gloves have two larger and significantly more powerful fans. One fan 3810 is located on the back of the hand portion of the glove and the other fan 3811 is located on the upper forearm sleeve area.

However, because A's breath doorbell receiver collects pathogens and has biosensor integration A's gloves is also rated to sanitize and disinfect his anti-pathogenic smart-home notification system.

A's neighbor D is an emergency room physician who also owns a pair of heavy-duty industrial strength anti-pathogenic gloves which are similar to A's gloves. However, D's gloves were slightly larger than A's gloves and constructed of more durable lightweight metal and carbon fiber. Additionally, D's anti-pathogenic gloves were longer than A's and covered more forearm area between D's wrist and elbow.

When D had demonstrated his antipathogenic glove system to A, A noticed that D's gloves had employed all of the same features and functionality as his (A's). However, the capabilities of D's gloves were more enhanced compared A's and had a higher environmental use certification than A's gloves.

When D had shown his gloves to A, A recalled that D's gloves also had three powerful internal fan systems on each glove. One fan was on the back of each hand and the other two fans were integrated with the gloves forearm extension.

Although A's gloves are also rated for industrial and some medical use applications, each glove had only two powerful fans. D's gloves also had a higher negative ionic energy output and exported a higher volume of air at a greater force output.

Additionally, both A and D's glove model are optionally capable of exporting heated air (like a hair dryer).

Like A's glove system, D's gloves were also upgraded with an optional ultraviolet light source (UV or UVX light spectrum source) and emitter lens for sterilizing germ-infested rooms and surface areas.

The UV light on both A and D's gloves could be turned off by a selector button on the forearm of the systems. D's gloves also had a slightly higher UV light output and were rated for use in emergency medical rooms and meat processing plants (EM-MPP).

D explained that his gloves were primarily designed to decontaminate emergency trauma and medical procedure rooms and were also rated and certified to use in meat processing plants where livestock, chicken and fish were processed for human consumption. Unlike A's gloves, D's gloves also had a temperature setting which controlled the temperature of the air exported from D's glove (like a hair dryer).

D had explained to A that unlike A's gloves, his (D's gloves) also contained an optional internal filtration system. D had shown A that his (D's) gloves also had a second negative ionic setting.

The second ionic setting is to prevent cross-contamination onto the glove. For example, it could be activated to neutralize the glove's air mass import and export vents (where contaminants settle after being imported and exported from the glove system).

D could also set the current to emit energy throughout the body of the glove to neutralize any surface contaminants which settled on the body of the glove (in the same manner in which germs are neutralized on the breath doorbell receiver).

In addition to the UV light source, in the palm of D's right glove was a circular spray nozzle cylinder. The nozzle is used to expel a germicidal spray solution from the antipathogenic glove onto contaminated surfaces to assist in surface disinfection.

A remembered that when D demonstrated by pressing a button on the forearm of the glove, it sprayed out a solution into the air (some models may initiate spray solutions by methods such as manual pumps, hydraulic forces or physical exertions such as thrusting or shaking an arm, etc.).

A put on the gloves, and turned them on as he walked towards the door to start disinfecting his system. C, his 10-year-old son who was doing homework at the kitchen table near B's sandwich, sneezed due to a cold.

A asked C to move away from the table. When C moved, A turned on the ‘IONS ONLY’ setting and began moving both hands 1-2 inches over the table to sanitize it. A also sanitized the area around B's sandwich. A turned off the ion setting and turned on the ‘FAN ONLY’ setting and starting blowing any remaining germs from the table. When A was finished, C sat back down to finish his homework.

A walked outside and again turned on the ‘IONS ONLY’ setting of his gloves and started sanitizing the area above and around the breath receiver device including the adjacent wall and door handle. A then tuned on the combined ‘FAN & ION’ setting for one minute. A then removed the gloves before going back inside.

### Description of A's Industrial Strength Anti-Pathogenic Gloves

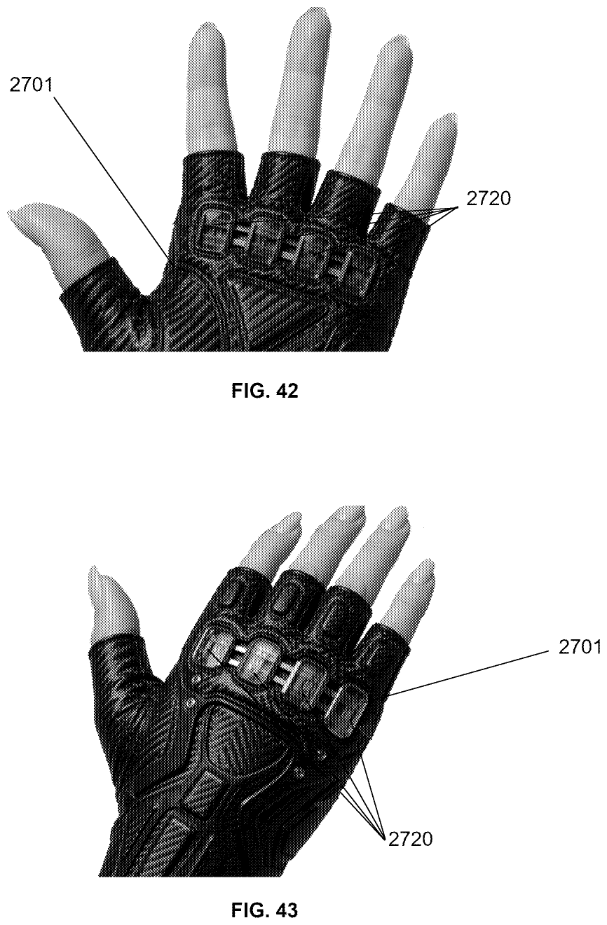

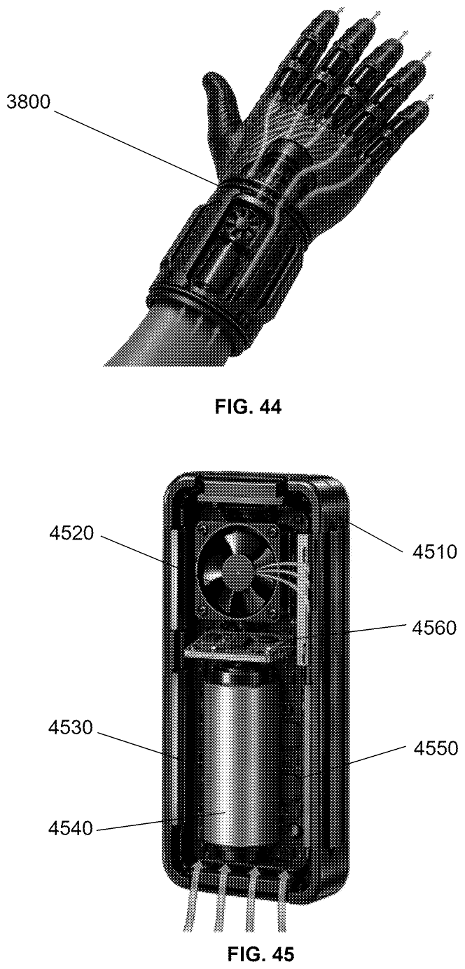

Additional details regarding the antipathogenic gloves 2700, 2701, and 3800 are illustrated in FIGS. 27, 27A, 28, 28A, 29, 38, 39, and 42-44. Use cases for the antipathogenic gloves are illustrated in FIGS. 30-36 and 40-41. A's industrial strength antipathogenic gloves 3800 employ all of the same features and functionality as his wife's (B's) antipathogenic gloves 2700 and 2701. However, as previously indicated, unlike B's light-weight INFANT-FORCE fingerless (2701) and full fingered (2700) gloves (having only one fan system (2710, 2711) on the back hand portion of each glove, A's industrial strength gloves have two larger and significantly more powerful fans 3810 and 3811.

A's gloves are high-tech with a sleek triangular import/export cone housing motor assembly case 3812 on the back of the hand. The triangular housing point peaks near the top center wrist portion of the glove.

The housing skirt (or triangle base) expands between the two furthest knuckles with an export vent 3813 positioned approximately over each of the four knuckles. Four additional export vents 3814 are integrated into the glove's palms and aligned approximately under the export vents on the back hand side.

This permits air and/or negative ionic energy to be forced outward between the hard layered hollow material (the air pathway) within A's glove layers and blow out of the glove knuckles and palm export vent openings. The expelled air pushes away germs and contaminants from surfaces subject to contamination.

Additionally, A's upgraded gloves came equipped with eight optional sliding vent shields 3815 (like a sliding shield covering a window inside a plane) over each export vent (3813, 3814) on the knuckles and within the open hand area above the palm of each glove.

Similar to air vents inside a vehicle the opening and closing of each vent redirects and increases air mass from one area of the glove to the various other export vents integrated within the antipathogenic gloves system.

Air is imported into the fan assembly from the two import openings. As shown, debris screens 3816 cover all import vents. In an embodiment, debris screens may also cover export vents. In an embodiment, the flexible joint area around A's thumbs and knuckles are made of flexible Kevlar. This allows A's fingers to move freely about under the carbon fiber hard shell and Kevlar covering.

The glove's internal components are illustrated in part in FIG. 45, and are covered with a carbon fiber hard shell or metal housing 4510, and include: an import/export system, at least one fan 4520, a power source such as a battery cell (can be chargeable) 4530, one or more motors to run the fan(s) 4540, an optional ion circuit configured to generate ions for decontamination 4560, and a communication module configured to communicate with anti-pathogenic primary and subsystem devices and system extensions 4550, as described herein.

In an embodiment, the ion circuit may also be configured to prevent cross-contamination onto the glove. For example, the ion circuit could be configured to neutralize the glove's air mass import and export vents (where contaminants settle after being imported and exported from the glove system).

The glove may also contain a motion detection circuit(s) such as an accelerometer or other movement detection means configured to automatically turn the device on and off based on the movement of the glove.

In an embodiment, the glove has an optional filtration system.

More advanced anti-pathogenic glove systems designed for industrial commercial or medical use applications may integrate U/V and/or UVC light energy system components and germicidal spray solutions into the glove system to enhance the glove's decontamination system.

The internal layers of the glove include at least some parts that are hollow. This formation allows high pressure air mass to flow between the inner and outer hardshell composite material of the contoured glove. The air mass is discharged (exported) from port openings along the open hand, palm, top knuckles, lower back of the hand or from the glove's fingers.

In an exemplified embodiment, similar to an air-conditioning vent inside a car, A's gloves may have upgraded opening and closing port covers to increase or decrease air pressure concentrations and/or ion energy output to preferred areas of the glove.

In an embodiment, the glove may include a feature or component that emits UV and/or UVC light at an intensity sufficient to kill pathogens. The light may be focused onto a desired target using a lens.

In an additional embodiment, the glove may include a heater configured to heat the expelled air mass. For example, the air may be heated to at least between 140 and 150 degrees, which is considered hot enough to kill bacteria, viruses, and other pathogens.

In an embodiment, the glove may include a spray mechanism configured to spray a disinfectant, which can be activated by pressing a button or other means.

### Embodiment 25—Anti-Pathogenic Glove Sprays UV Light & Heat Integration

D is a heart surgeon who works at a hospital. D was assigned to the level-1 emergency trauma room. After treating a gunshot victim, D noticed new physician intern F walk into the room.

F was wearing an industrial strength antipathogenic glove system on his right hand. It was similar to the one D had demonstrated to A earlier. However, the glove system F had was yellow.

D asked F where he had got the new glove system. F told him two gloves were purchased by the hospital and assigned to the emergency room due to higher levels of pathogen and germ exposure than other parts of the hospital. The gloves were purchased for medical techs and nurses to use to decontaminate beds, rooms and medical devices after patients are cleared from the room after procedures.

F explained this system was the newest and most advance model which had been released. F had already seen the system D had.

F told him that his model (the hospital's) had all the features and functionality of D's antipathogenic glove system but had more technology. F told D that the glove had the ability to identify and neutralize germs using UVC and UV spectrum light.

F demonstrated by turning off the lights in the emergency room. F then held the palm lens over a counter where several strains of germs were observed under the light. F used the UV light of the glove to neutralize the germs.

F also showed D an air temperature setting on the glove forearm. F demonstrated further by turning the glove on so that it exported heated air. F then set the power level to 1500 watts (similar to a hair dryer), increasing the temperature of the expelled air to 160 degrees, which is hot enough to kill most pathogens and bacteria.

After the germs were neutralized F pressed on a small circular aperture extension attached to the inside wrist of the glove, causing it to spray a germicidal solution to assist in surface decontamination.

FIG. 27 illustrates an open palm view of a standard antipathogenic glove system device which may be worn as a piece of personal protective equipment (PPE) for everyday wear (or as fashion attire) as a person might wear a multi-use or disposable mask. There are 4 export vent openings 2720 with manual slide coverings (shown opened) integrated within the upper palm of the glove hand.

The vents have an optional shield and latch 2725 which can be closed or opened to proportionally or disproportionally deliver air mass concentrations and ionic energy from different parts of the glove system to preferred areas of the glove for exportation or emission onto surfaces or into contaminated areas to neutralize the germs and contaminants.

The air mass is optionally temperature controlled to neutralize airborne pathogens and contaminated surfaces. In FIG. 27, the glove has a removable cloth material surface which stretches across a hollow carbon fiber exoskeleton (not shown) frame. However, the material can alternatively be constructed of various types of hardened materials, including plastics and metals. Air which is imported into the glove is transferred through the exoskeleton and into a fan charging system.

In an embodiment, the air mass is infused with negative ions before being pushed through the exoskeleton and out the export vents 2720. However, in other embodiments, the exported mass and emissions are not limited to transference through the hollow exoskeletons.

For example, in a second glove embodiment, import and export vents may be independently separate, affixed or detachable from or to any portions of the glove hand and the mass may also originate from a separate, affixed or detachable portion (in essence-mini subsystems-as defined in the granted parent application) of the glove system frame or exoskeleton. The exoskeleton may not even be hollow with no need for mass transference through glove framing.

FIG. 28 illustrates a top-down view of the antipathogenic glove system device 2700. As an alternative to standard fan propulsion technology, the propulsion mechanism for the antipathogenic system may employ a bladeless motor system or brushless fan system technology (or a combination thereof, but more conveniently integrated into large industrial strength equipment type gloves).

There are 4 additional air mass export vents 2720 configured to operate in the same manner as the vents shown in FIG. 27. The export vents sit over or proximate to the knuckles and discharge air and ionic energy or ions out of the vents. The vents can be slid open or closed to deliver air mass concentrations and ionic energy from preferred areas of the glove onto surfaces or into contaminated areas to neutralize germs and contaminants. Import vents 2730 are integrated into each side of the triangular fan motor assembly housing. However, various propulsion designs are possible.

The import vents are also ported into the engine intake mechanism where the air is charged, circulated and optionally ion infused for delivery into the hollow exoskeleton and out the export openings. The motor assembly contains a negatively charged ion wire and generator. The wire is preferentially positioned within the import and/or export pathways of the hollow exoskeleton to assure force from the fan system pushes or pulls ions through the glove at a sufficient speed to be disbursed over a wide area.

The gloves 2700 and 2701 may include a motor assembly, a fan, an optional motion detector, a charging port, control buttons (e.g., speed control, on/off switch), an ion generator, a communication module or chip, fan speed controller circuit, and a power source, e.g., a battery. Larger more advanced antipathogenic glove systems (i.e. industrial strength models) may optionally include UV/UVC light emitters, UV-based germ detectors, filters, germicidal spray systems, and/or heat generators.

FIGS. 27A and 28A illustrate, respectively, an open palm view and a top-down view of a fingerless antipathogenic glove system device 2701. The fingerless glove may be worn as a piece of personal protective equipment (PPE) and for coddling infants and babies. The fingerless glove has the same features and functionality of the glove 2700 illustrated in FIGS. 27 and 28, except that it is fingerless to ensure physical contact between a mother and her baby.

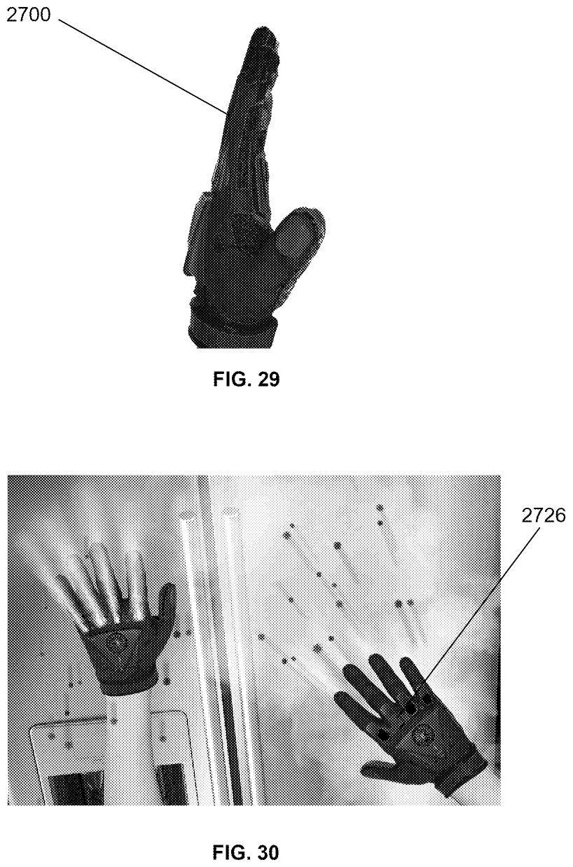

FIG. 29 illustrates a side view of the antipathogenic glove system device 2700. It shows the raised fan motor housing (not to scale) with import vent integration.

FIG. 30 illustrates one of several applications the technology can be used for. The FIG. 30 representation does not distinguish between specific types of antipathogenic glove use application (i.e. industrial, maternity, fashion PPE etc.), but presents some general capabilities of the technology. In FIG. 30, the user is sterilizing a refrigerator surface. The refrigerator may be inside a residential or commercial restaurant kitchen.

The person sanitizing the refrigerator is using a combination of hand motions to wipe over the surface area without necessarily coming in physical contact with the appliance. The right glove is exporting air (represented by blue) from the palm vents. Air is coming from all vents within the palm and from the vents in the knuckles, except the middle finger vent 2726 which is closed. Because this vent is closed, less force is coming from the knuckle vents and more air is being diverted to the adjacent knuckle and palm export vents.

Pathogens (red dots) and germs (black dots) are being pushed away from the surface area of the refrigerator. Surface sterilization can occur prior to or after the appliance has already been physically cleaned by other means. The user's left hand in FIG. 30 is emitting negative ionic energy (represented by green) which is being exported from the glove knuckle vents. The concentration of ionic energy escaping from the glove is high. This is because no energy is escaping from the bottom of the glove, which indicates all palm vents are closed.

The ions are being used to kill pathogens and germs on the refrigerator in conjunction with the exported air.

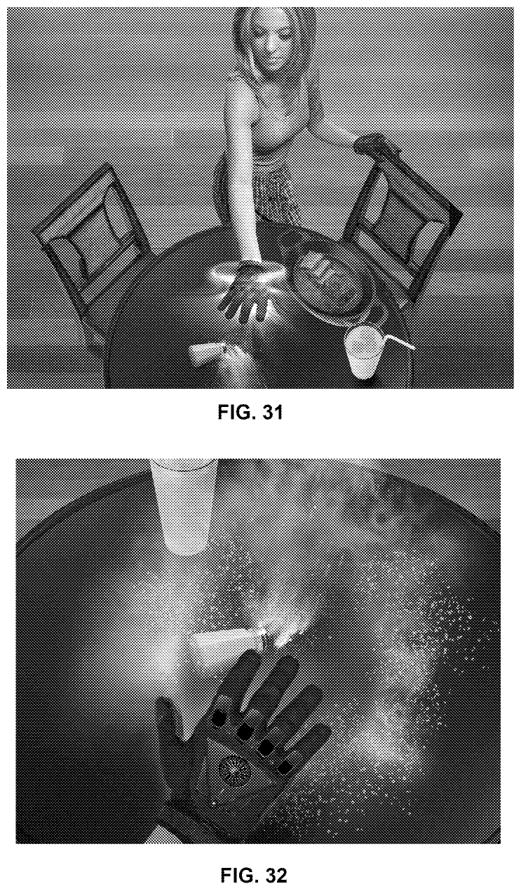

FIG. 31 illustrates another use application for the glove. In FIG. 31 the user has turned on only one of her standard strength antipathogenic gloves, but has activated both the air and ion setting. She turned on the air function to blow away a small amount of salt that her child had dumped over the kitchen table.

Although the glove will blow the salt onto the floor, she plans to clean the floor after breakfast. Her child was recently diagnosed with bacterial strep throat. Because he had sneezed at the table before spilling the salt, she had also decided to ionize the table and food that he left.

FIG. 32 illustrates a close-up view of negative ions emitting from the glove and onto the table. However, the blue air function has been turned off. The user in FIG. 32 had turned on the negative ion function to neutralize any surface germs which had been left on the table after her child had sneezed.

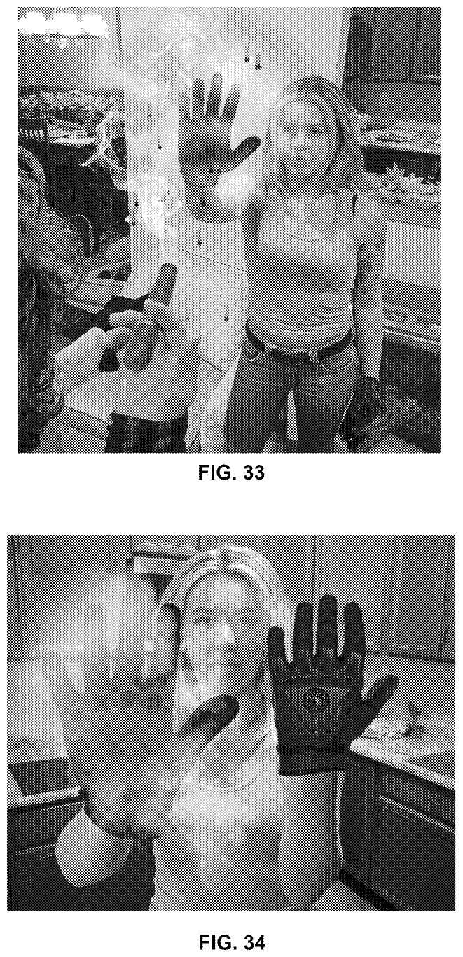

FIG. 33 illustrates a user who has activated the air and negative ion function on her antipathogenic glove. She uses the air to push away carcinogenic cigarette smoke. The glove also emits (and blows) ionic energy towards the source of the smoke to assist in neutralizing the airborne toxic (falling black dots) contaminants.

FIG. 34 illustrates a user wearing two antipathogenic gloves. All knuckle vents are closed on her left glove so that the negative ions (green) from the glove are all being used to self-disinfect her body. She is self-disinfecting because she is being exposed to a sick person (not visible in FIG. 34). She is simultaneously using air mass (blue) from her right glove to push foul body and breath odor away from her face, originating from the same person in partial view in FIG. 33.

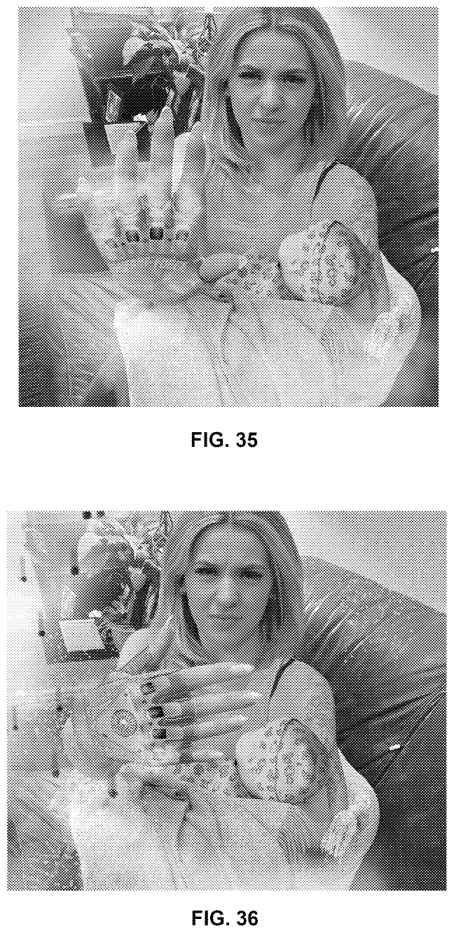

FIG. 35 illustrates a user who has purchased a pair of fingerless antipathogenic gloves for infants. In FIG. 35, because her infant is a newborn she does not want to use the negative ion setting. However, she activates the blue air function on her glove with all palm vents open to maximize air force and push germs away from her and her newborn when her other two children (not in view) walked into the house coughing.

In FIG. 36, more than a year and a half has passed since the embodiment in FIG. 35. In FIG. 36, the mother is again coddling her toddler. She was breast-feeding on the couch when her two sick children walked inside the home coughing. Her pediatrician told her it was now ok to occasionally use the negative ion setting around her baby.

The mother turned on both, the air and ion setting. She shielded her baby, using the combined protection of air and ions. She kept her hand stationary in placed out in front of her and her baby as her younger children walked closer. In doing this, she created a partial forcefield around them against encroaching germs. In FIG. 36, red pathogens and black germs are observed dropping after being neutralized and pushed away.

FIG. 37 illustrates a self-sanitizing breath doorbell bio receiver. The outer circular ring trim 3710 is green indicating the system is emitting negative ionic energy. However, ionic energy is also infused throughout the receiver, including the center filament where most of a person's breath sample is captured.

The energy kills germs resting on the surface and helps prevent cross contamination when the device is touched or handled when maintenance is performed. This is because germs which have settled on the bio receiver during sample collection process have already been neutralized by ionic energy searing throughout the receiver.

Alternatively, direct electrical energy pulses can also be used in bio receivers to kill airborne viruses and bacteria which have previously settled on the receiver surface and surfaces adjacent to receiver integration points.

FIG. 38 illustrates a top-down view of an industrial strength embodiment 3800 of the antipathogenic glove device. It has the same basic features and functionality of the non-industrial models 2700 and 2701. However, because of its larger size (the forearm extension allows for more carry space) it can enclose more technologies while still maintaining an aesthetic shape receptive to human hand and arm motions. Industrial strength antipathogenic glove system devices may also be programmed to communicate with other antipathogenic systems as described herein.

The industrial strength gloves 3800 are more readily associated with wearable, heavy duty pieces of equipment than the antipathogenic gloves system devices 2700 and 2701 due to increased size, power and load capacity. The propulsion mechanism for industrial glove system 3800 may also employ bladeless fan or brushless motor system technology (or a combination thereof).

The hand portion of the glove 3800 forward of the wrist has the same basic functionality and design as antipathogenic gloves 2700 and 2701. However, the industrial strength glove 3800 has a significantly stronger motor, fan and energy output in the glove hand than gloves 2700 and 2701. The integrated forearm extension in glove 3800 also has increased load bearing and power mechanisms and may employ multiple fan systems and additional technological capabilities.

The broken blue line 3818 under the right import vent 3820 on the hand represents air, while the broken green line 3817 under the left import vent 3819 of the hand represents ionic energy. The broken yellow line 3821 on top of the forearm is the universal sign for caution and signals that industrial strength glove equipment may employ UVX, UVC, and UV spectrum light and/or germicidal spray capabilities potentially harmful to humans at unregulated amounts and intensities.

The wrist portion of the exoskeleton is unified into the forearm encasement from where additional air and energy is charged and infused from the second fan system (the system may have several fans). The additional air is then forced through the frame or exoskeleton of the glove, where it is released from the hand vents and back into the environment to neutralize surfaces and areas subject to contamination.

The export vents 3813 (closed dark red vents 3815 on the knuckles) can be slid open or closed to deliver calculated amounts of air mass and ionic energy from preferred areas of the glove onto surfaces or into germ compromised areas at higher velocities and emissions to neutralize contaminants.

The fan import vents (3819, 3820) are on top of the forearm encasement between the forearm fan 3816 and wrist. However, various propulsion and design configurations are possible regarding antipathogenic system gloves and extension devices.

The glove 3800 may include a motor assembly, one or more fans, an optional motion detector, a charging port, exterior control buttons (e.g., a fan speed controller, an on/off switch, mode switch, etc.), an ion generator, a communication module, a fan speed controller, and a power source such as a battery.

Larger more advanced antipathogenic glove systems may optionally include motion detecting accelerometers or other motion detecting components, UV/UVC light emitters, UV-based germ detectors, filters, germicidal spray solution systems, and/or heat generators.

The import vents are also ported into the engine intake mechanism where the air is circulated, charged and optionally ion infused for delivery into the hollow exoskeleton and out the export openings in the glove hand.

The motor assembly contains a negatively charged ion wire and generator. The wire is preferentially positioned within the import and export pathways of the hollow exoskeleton to assure force from the fan system pushes or pulls the ions through the glove at a sufficient speed to be disbursed over a wide area as it exits the glove.

FIG. 39 illustrates an open palm view of the industrial strength antipathogenic glove system device 3800 with UVX, UVC, or UV light bar lens 3830 under the palm export vents 3814. FIG. 39 also shows an integrated circular, germicide spray aperture attachment 3840 in the palm of the glove. The glove 3800 may also be worn as a piece of personal protective equipment (PPE) and to sanitize and decontaminate environments and objects prone to heavy bacterial and pathogen contamination.

Some of these environments include: medical trauma rooms, doctors'offices (i.e. after successive patient visits), hospital patient rooms, meat, seafood and chicken processing plants and preparation facilities, commercial scale kitchen appliances and food storage facilities, commercial passenger airplanes, cruise ships (i.e. before and after passenger boarding and debordering) and public transportation system vehicles such as: buses, trains, cabs etc. FIG. 39 shows four export vents 3814 in the palm of the glove in the open position.

The UV light 3830 can be turned on or off by a selector button on the forearm control panel. The wearer can hold his gloved hand over bacteria and/or virus infected surfaces to neutralize or kill these contaminants. Some models may also employ ultraviolet light lens emitters (not shown in FIG. 39) to identify surface bacteria and pathogen adhering to surfaces subject to decontamination.

FIG. 40 illustrates a user employing an industrial strength antipathogenic glove system device 3800. The user is wearing it on his left hand with his palm open. He is holding the glove approximately 2-3 inches away from and over an antipathogenic breath doorbell bio receiver 3700. The green energy emitting from the glove and saturating the breath doorbell represents negative ionic energy emitting from the glove and onto the receiver. In this embodiment, the doorbell receiver 3700 is not equipped with a negative ion neutralization system, and so the user is decontaminating the receiver with his glove. Black germs and red pathogens are depicted falling down from the breath doorbell receiver after being neutralized.

FIG. 41 illustrates a user who is wearing two industrial strength antipathogenic glove system devices. However, the user has just completely turned off his left glove. The glove on the user's right hand is disbursing and emitting a powerful, temperature-controlled mixture of forced hot air (blue) and negative ions (green) to decontaminate the audio-visual home vehicle visitor alert system. The neutralized germs (black) and pathogens (red) are being forced away from the contaminated air around the glove and from the surface of the device.

Any of the embodiments disclosed herein may include one or more of the following option components: an ion generator configured to generate ions for the elimination and/or dispersal of pathogens; a UV and/or UVC generator configured to generate energy for the elimination and/or dispersal of pathogens; a UV and/or UVC generator configured to generate energy for the identification of pathogens; a heater configured to increase the temperate of surrounding or expelled air for the purpose of elimination and/or dispersal of pathogens; and a spray mechanism configured to spray a disinfectant for the elimination of pathogens.

### Mobile Docking Pediatric Field Embodiment

In additional embodiments, the containment system is configured to be dockable with permanent docking containment infrastructure. In a particular embodiment, a docking containment system is configured for pediatric deployment, including use within pediatric hospital wings, neonatal transport corridors, emergency intake areas, temporary field units, and mobile medical environments.

In this embodiment, a pediatric patient positioned on an occupant support surface (e.g., a hospital bed) is transported into alignment with a mobile docking containment structure. The docking structure defines a containment region scaled for pediatric bed dimensions while maintaining protective airflow boundary formation around at least a portion of the occupant support surface.

The docking containment structure may be further configured to:
provide adjustable airflow intensity appropriate for pediatric physiology;
allow adjustable and/or programmable airflow trajectory;
reduce airflow velocity while maintaining outward boundary integrity;
provide ionization or contaminant-reduction levels adjustable for neonatal or immunocompromised pediatric patients; and/or
allow rapid docking and undocking during transport between treatment zones.

Unlike fixed architectural containment rooms, the mobile docking configuration enables:
deployment without structural renovation;
increased structural stability;
reduced reliance on disposable enclosure materials;
improved caregiver access without dismantling barriers;
configurable room-based airflow containment zones.
reduced setup time;
rapid repositioning between rooms;
use in temporary care areas;
scalability for surge events; and
continuity of protective airflow during intra-hospital transport.

In one embodiment the docking containment structure may function as part of a designated room or containment environment intended for immunocompromised, neonatal, infectious disease, or other protected-care applications. In certain embodiments, the docking configuration may provide a more open and observable patient environment compared to traditional fabric isolation tents, which may help reduce claustrophobic effects while also improving caregiver visibility and access.

The docking system may be configured to operate independently of permanent room infrastructure and may be powered by wall power, battery systems, vehicle power, or hybrid sources.

In an embodiment, the docking containment structure interfaces with permanent or semi-permanent room infrastructure. For example, the docking containment structure may align with, couple to, or communicate with airflow ports, recessed channels, wall-mounted airflow systems, filtration systems, ionization systems, or environmental control systems integrated into a wall, architectural structure, or room structure.

In some embodiments, the docking region itself may be partially or fully integrated into a wall or architectural structure. For example, a recessed or surface-mounted geometric boundary region (e.g., rectangular, circular, polygonal, curved, or other shapes) may define one or more airflow emission zones configured to direct airflow outward around a patient-support surface in order to form a protective airflow boundary.

The docking structure may also mechanically or pneumatically interface with the room structure. For example, hooks, rails, latches, couplers, magnetic interfaces, alignment structures, airflow conduits, flexible ducts, or telescoping airflow transfer members may connect the docking structure to the wall-mounted system or architectural airflow source.

In some embodiments, the docking structure may remain mobile and repositionable. In other embodiments, the docking structure may be stationary, wall-associated, partially recessed, permanently installed, or integrated into architectural infrastructure while still maintaining the protective airflow boundary functionality.

The containment docking system has many benefits in comparison to temporary isolation tent structures.

Traditional isolation tents typically: rely on passive enclosure barriers, require full fabric encapsulation, restrict caregiver access, may require zipper or flap entry, may create visual obstruction, and/or may rely primarily on negative-pressure containment without dynamic outward boundary emission.

By contrast, the docking containment system:
forms an outward protective airflow boundary without requiring full fabric encapsulation;
allows rapid caregiver access without dismantling barriers;
permits improved visual clarity;
may reduce physical contact with enclosure surfaces;
may operate independently of tent structures or permanent infrastructure;
may escalate airflow intensity during outbreak conditions while maintaining adjustability for patient comfort.

In mobile field applications, the docking containment structure may provide enhanced convenience relative to tent-based isolation by:
allowing rapid repositioning;
reducing setup time;
eliminating reliance on extensive fabric structures;
providing configurable structural modules;
enabling deployment in hospital rooms, transport vehicles, surge facilities, or temporary installations.

The containment docking system may also be utilized in combination with tent environments, or as an alternative thereto, while maintaining airflow boundary protection around at least a portion of the occupant support surface.

In an additional use case for the docking containment system, a pediatric patient initially positioned within a traditional isolation tent may be transitioned to a docking containment system without requiring bed replacement.

The docking containment structure may be positioned relative to the existing occupant support surface, allowing:
removal of tent fabric structures;
preservation of medical equipment positioning;
improved caregiver access;
improved emergency responsiveness;
maintenance of airflow-based boundary protection.

This configuration may reduce logistical burden during pediatric transport, surge events, or temporary facility deployments.

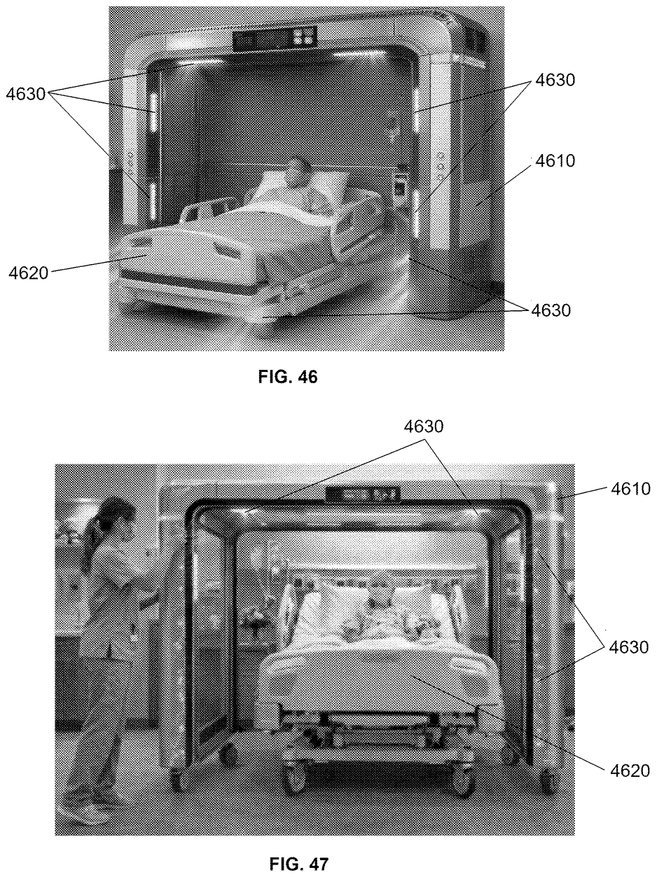

With reference to FIGS. 46 and 47, an example anti-pathogenic docking containment system includes a structural frame or module 4610 defining a containment region around an occupant support surface, such as a hospital bed 4620; and at least one airflow generation subsystem 4630 operatively associated with the structural frame and configured to emit airflow outward along at least one boundary of the containment region to form a protective airflow boundary around at least a portion of the occupant support surface.

The structural frame 4610 is operable and moveable independent of the occupant support surface 4620. The structural frame or module may be configured to be integrated with, attached to, or detachable from the bed or occupant support surface. The structural module may be configured for one or more of articulated, pivotable, translatable, sliding, rotational, and angular repositioning relative to the occupant support surface.

Furthermore, the structural frame 4610 is configurable to be docked with a more permanent infrastructure element, such as a wall structure, architectural structure, room structure, building structure, mobile platform, or vehicle structure. The docking function may be achieved by any presently known or future means of docking, and may include one or more of integration into, mounting to, or positioning relative to the infrastructure element.

In an embodiment, the structural frame or module is reconfigurable between a bed-associated configuration and a remote-mounted configuration while maintaining airflow containment capability.

A remote-mounted configuration includes embodiments where the containment structure and/or airflow generation subsystem is positioned separately from direct attachment to the bed or occupant support surface, while still functionally cooperating with or defining a protective containment region relative to the occupant support surface.

For example, the remote-mounted configuration may include wall-mounted, recessed, ceiling-mounted, architectural, or infrastructure-integrated embodiments that remain aligned with, docked relative to, or positioned adjacent to the occupant support surface while maintaining airflow containment capability.

In some embodiments, the docking relationship may occur through physical attachment, alignment, proximity positioning, airflow coupling, telescoping engagement, rail systems, hook systems, conduit coupling, recessed interfaces, or other structural and/or airflow communication mechanisms.

The system may transition between a bed-associated or directly attached configuration and a remote-mounted or infrastructure-associated configuration, while maintaining at least partial protective airflow boundary functionality around the occupant region.

The airflow emitted by the airflow generation subsystem(s) 4630 may be configured to emit laminar airflow, turbulent airflow, directional airflow, directional airflow of adjustable trajectory, or combinations thereof. The airflow generation subsystem(s) are attached to, or otherwise associated with, the structural frame and/or the bed or occupant support surface in such a manner as to enable adjustable outward airflow trajectory to resist encroachment of airborne contaminants.

In certain embodiments, the docking containment structure may cooperate with, supplement, align with, or operate concurrently with airflow emitted from the bed or occupant support surface itself.

For example, the occupant support surface may independently generate airflow, directional airflow, laminar airflow, ionized airflow, or contaminant-reduction airflow, while the docking structure provides additional airflow containment boundaries around the occupant region.

In some embodiments, the docking structure may operate independently of the bed airflow system. In other embodiments, the docking structure and occupant support surface may function cooperatively, simultaneously, sequentially, or in synchronized containment modes.

These embodiments utilize airflow generated from both the docking structure and the occupant support surface together to form a larger or more dynamic protective airflow boundary around the patient.

Any of the elements of any embodiment disclosed herein may be incorporated into the dockable embodiment(s) as may be useful and/or desired, and any and all such combinations are within the scope of the present invention.

Embodiments of the subject matter and the functional operations described in this specification can be implemented in one or more of the following: digital electronic circuitry; tangibly-embodied computer software or firmware; computer hardware; and combinations thereof. Such embodiments can be implemented as one or more modules of computer program instructions encoded on a non-transitory medium for execution by a data processing apparatus.

As used herein, the term “data processing apparatus” comprises all kinds of apparatuses, devices, and machines for processing data, including but not limited to, a programmable processor, a computer, and/or multiple processors or computers. Exemplary apparatuses may include special purpose logic circuitry, such as a field programmable gate array (“FPGA”) and/or an application specific integrated circuit (“ASIC”). In addition to hardware, exemplary apparatuses may comprise code that creates an execution environment for the computer program (e.g., code that constitutes one or more of: processor firmware, a protocol stack, a database management system, an operating system, and a combination thereof).

The term “computer program” may also be referred to or described herein as a “program,” “software,” a “software application,” a “module,” a “software module,” a “script,” or simply as “code.” A computer program may be written in any programming language, and it can be deployed in any form, including as a standalone program or as a module, component, subroutine, or other unit suitable for use in a computing environment. A computer program can be deployed and/or executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, such as but not limited to an FPGA and/or an ASIC.

Computers suitable for the execution of the one or more computer programs include, but are not limited to, general purpose microprocessors, special purpose microprocessors, and/or any other kind of central processing unit (“CPU”).

Computer readable media suitable for storing computer program instructions and data include all forms of nonvolatile memory, media, and memory devices. For example, computer readable media may include one or more of the following: semiconductor memory devices, such as ROM or RAM; flash memory devices; magnetic disks; magneto optical disks; and/or CD-ROM and DVD-ROM disks. The processor and the memory can be supplemented by, or incorporated in, special purpose logic circuitry.

To provide for interaction with a user, embodiments may be implemented on a computer having any type of display device. Exemplary display devices include, but are not limited to one or more of: projectors, cathode ray tube (“CRT”) monitors, liquid crystal displays (“LCD”), light-emitting diode (“LED”) monitors, and/or organic light-emitting diode (“OLED”) monitors. The computer may further comprise one or more input devices by which the user can provide input to the computer. Input devices may comprise one or more of: keyboards, pointing devices (e.g., mice, trackballs, etc.), and/or touch screens. Moreover, feedback may be provided to the user via any form of sensory feedback (e.g., visual feedback, auditory feedback, or tactile feedback). A computer can interact with a user by sending documents to and receiving documents from a device that is used by the user.

Embodiments of the subject matter described in this specification can be implemented in a computing system that includes one or more of the following components: a backend component (e.g., a data server); a middleware component (e.g., an application server); a frontend component (e.g., a client computer having a graphical user interface (“GUI”) and/or a web browser through which a user can interact with an implementation of the subject matter described in this specification); and/or combinations thereof. The components of the system can be interconnected by any form or medium of digital data communication.

The computing system may include clients and/or servers. The client and server may be remote from each other and interact through a communication network. The relationship of client and server arises by virtue of computer programs running on the respective computers and having a client-server relationship to each other.

Various embodiments are described in this specification, with reference to the detailed discussed above, the accompanying drawings, and the claims. Numerous specific details are described to provide a thorough understanding of various embodiments. However, in certain instances, well-known or conventional details are not described in order to provide a concise discussion. The figures are not necessarily to scale, and some features may be exaggerated or minimized to show details of particular components. Therefore, specific structural and functional details disclosed herein are not to be interpreted as limiting, but merely as a basis for the claims and as a representative basis for teaching one skilled in the art to variously employ the embodiments.

The embodiments described and claimed herein and drawings are illustrative and are not to be construed as limiting the embodiments. The subject matter of this specification is not to be limited in scope by the specific examples, as these examples are intended as illustrations of several aspects of the embodiments. Any equivalent examples are intended to be within the scope of the specification. Indeed, various modifications of the disclosed embodiments in addition to those shown and described herein will become apparent to those of ordinary skill in the art, and such modifications are also intended to fall within the scope of the appended claims. Furthermore, any reference to singular includes plural embodiments, and any reference to more than one component may include a singular embodiment.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple products.

All references including patents, patent applications and publications cited herein are incorporated herein by reference in their entirety and for all purposes to the same extent as if each individual publication or patent or patent application was specifically and individually indicated to be incorporated by reference in its entirety for all purposes.

What is claimed is:

1. An anti-pathogenic docking containment system comprising:
a structural frame defining a containment region positionable relative to an occupant support surface; and
at least one airflow generation subsystem operatively associated with the structural frame and configured to emit airflow outward along at least one boundary of the containment region to form a protective airflow boundary around at least a portion of the occupant support surface;
wherein the docking containment system is operable independently of the occupant support surface;
wherein the structural frame is configurable for integration into, mounting to, or positioning relative to a wall structure, architectural structure, room structure, building structure, mobile platform, or vehicle structure; and
wherein the airflow generation subsystem is configured to emit laminar airflow, turbulent airflow, directional airflow, directional airflow of adjustable trajectory, or combinations thereof.

2. An anti-pathogenic modular containment apparatus comprising:
a structural module configured to be integrated with, attached to, or detachable from a bed or occupant support surface; and
an airflow generation subsystem associated with the structural module and configured to emit outward airflow to resist encroachment of airborne contaminants;
wherein the structural module is reconfigurable between a bed-associated configuration and a remote-mounted configuration while maintaining airflow containment capability; and
wherein the structural module is configured for articulated, pivotable, translatable, sliding, rotational, angular repositioning, or combinations thereof relative to the occupant support surface.
